(19) United States
(12) Patent Application Publication
(10) Pub. No.: US 2026/0335572 A1
(43) Pub. Date: Oct. 29, 2026

(54) AIR-JET DRY POWDER INHALER (DPI) WITH PASSIVE CYCLIC LOADING OF THE FORMULATION

(71) Applicant: VIRGINIA COMMONWEALTH UNIVERSITY, RICHMOND, VA (US)

(72) Inventors: Philip Worth LONGEST, RICHMOND, VA (US); Connor HOWE, RICHMOND, VA (US); Dale FARKAS, RICHMOND, VA (US); Michael HINDLE, RICHMOND, VA (US); Mohammad MOMIN, RICHMOND, VA (US)

(21) Appl. No.: 19/104,883

(22) PCT Filed: Aug. 23, 2023
(86) PCT No.: PCT/US2023/030937

§ 371 Date: Feb. 19, 2025

Related U.S. Application Data
(60) Provisional application No. 63/373,251, filed on Aug. 23, 2022.
(60) Provisional application No. 63/373,251, filed on Aug. 23, 2022.

(51) Int. Cl. A61M 15/00

(52) U.S. Cl. CPC A61M 15/0065; A61M 15/0005; A61M 2202/064

(57) ABSTRACT

Dry powder inhalers (DPIs) and related platforms are disclosed with a passive cyclic loading mode of operation. Metering elements such as a powder shelf may be used to control the amount of powder that is aerosolized with each actuation. Advantages of the passive cyclic loading system include metering a consistent amount of powder for each actuation while protecting the powder in the reservoir from aggregate formation. A single device can accommodate a variable range of powder doses and aerosolize a relatively consistent amount of dose with each actuation, while remaining insensitive to the amount of powder loaded (above a certain threshold).

### CROSS-REFERENCE TO RELATED APPLICATIONS

This application claims the benefit of U.S. Provisional Patent Applications 63/373,251, filed Aug. 23, 2022, and 63/466,777 filed May 16, 2023. The complete contents of these applications is incorporated herein by reference.

### STATEMENT REGARDING FEDERALLY SPONSORED RESEARCH AND DEVELOPMENT

This invention was made with government support under Contract Nos. 5R01HL139673 and 5R01HD087339 awarded by the National Institutes of Health. The US government has certain rights in the invention.

### FIELD OF THE INVENTION

Embodiments generally relate to dry powder inhalers (DPIs) and, more particularly, devices and methods for efficient delivery of formulations to patients such as infants.

### BACKGROUND

Delivery of pharmaceutical aerosol at high doses to infants, children, and adults is associated with significant challenges, most notably poor lung delivery efficiency, high intersubject dose variability, and long administration times. Poor lung delivery efficiencies of aerosols have been documented in most in vivo and realistic in vitro infant studies, which typically report lung delivery efficiency values in the range of 0-10% of the initial dose across multiple inhalation platforms. Low lung delivery efficiency, high intersubject variability and extended aerosol delivery times may all contribute to poor therapeutic outcomes and, for pharmaceuticals with narrow therapeutic windows, significant off-target effects.

WO2021/150883A1 discloses several multidose storage and delivery devices (MDUs). In one MDU, a dose release selector allows a user to manually adjust which of several subchambers filled with powder open and which are closed. A drawback of this design is the necessity of active reloading. A user or motor must physically turn a retaining disk to open and close the subchambers. Further MDUs employ barriers containing one or more small openings sized to limit or prevent passage of powder solely under the force of gravity. Instead of releasing powder prior to inhaler actuation, these MDUs dose masses of powder to an aerosolization chamber during inhalation actuation. The small air currents during inhaler actuation entrain powder from within the MDU and drive it into the aerosolization chamber and onward to the patient. Though this passive reloading approach is beneficial for some applications, it has certain drawbacks. For instance, the device may be sensitive to downstream pulmonary mechanics. The dose for a single inhalation may depend to a not insubstantial degree on the volume of gas used for the inhalation and the rate of its delivery. Freely selecting dose per inhalation, inhalation volume, and delivery rate independent of one another is not readily possible.

Even in adults, the availability of high dose DPIs is limited. Some DPI devices administer>10 mg of drug. However, these devices are designed to operate with very large inhaled air volumes (on the order of ~2 L and above) and high airflow rates (typically 45 L/min and above). Moreover, they may incur significant extrathoracic depositional losses. Alternatively, higher dose delivery is often accomplished by requiring the patient to use a relatively low dose device and load multiple capsules with repeated inhalations over multiple cycles. This approach is far from ideal, since it increases the difficulty of device operation as well as the opportunity for user errors. Where infants are concerned, high dose DPI solutions are even more limited. With infants, it is not possible to resort to large inhaled air volumes (e.g., 2L and above) or high airflow rates (e.g., 45 L/min and above).

One option to increase the aerosolized dose of the infant air-jet DPI is the use of a larger aerosolization chamber to accommodate more loaded powder. However, increasing the infant air-jet DPI aerosolization chamber volume to accommodate higher mass loadings can negatively impact aerosol performance, especially when only 10 mL of air is available to aerosolize the powder. Irrespective of the size of the aerosolization chamber, an aerosolization chamber may simply be loaded with more powder (provided the size is at least large enough to accommodate the powder load volume). However, this solution can also be problematic, especially when the increased load necessitates multiple actuation cycles. Repeated exposure of dry powder formulations to aerosolization forces within the device creates aggregates that are then difficult to disperse with subsequent actuations.

### SUMMARY

To better address the challenges associated with pharmaceutical delivery to the lungs, especially those of infants and for high dose aerosol to all ages, some embodiments are directed to air-jet dry powder inhaler (DPI) platforms and related methods for rapid and efficient aerosol administration, e.g., nose-to-lung.

An exemplary platform may include an air source (providing aerosolization energy and inhalation gas), an air-jet DPI (responsible for holding the loaded powder and aerosolizing metered doses per actuation), and a patient interface (which transports the aerosol from the DPI to the patient's airways).

Some embodiments focus in particular on the DPI component employable with delivery platforms. An exemplary air-jet dry powder inhaler (DPI) includes features enabling passive cyclic loading procedures to provide high efficiency aerosol lung delivery while being insensitive to powder mass loadings and the presence of downstream pulmonary mechanics.

An exemplary air-jet DPI may be a general aerosol delivery platform. An exemplary general high dose DPI platform allows for different powder mass loadings without significantly impacting performance or operation. Such a device permits methods of treatment which require multiple breaths to maintain an acceptable lung deposited powder mass with each inhalation. It is advantageous for an air-jet DPI to enable variable powder mass loadings, including loadings in excess of 10 mg for infants, without impacting the high lung delivery efficiency.

An exemplary air-jet DPI or general aerosol delivery platform may be employed for any of multiple applications. Use of a platform where high efficiency lung delivery of a pharmaceutical aerosol may be beneficial include, for example, administering: inhaled surfactant in surfactant replacement therapy, inhaled antibiotics to treat bacterial pneumonia, and inhaled antivirals to treat RSV and severe viral pneumonia. In each of these applications, relatively high doses of powder, typically in the range of 10 mg and above for an infant, are expected to be required to generate a sufficient biological response.

An exemplary method of delivering a full dose of powder to a patient using a dry powder inhaler (DPI) may comprise, repeating for consecutive cycles: pre-actuation, metering an amount of powder from a reservoir through an opening into an aerosolization chamber by positioning the reservoir above a powder support surface of the aerosolization chamber with respect to gravity so that a metering space between the powder support surface and the opening fills with powder of an amount less than the full dose; actuating the DPI so that one or more air jets pass through the aerosolization chamber to aerosolize the powder; and transporting the aerosolized powder from the aerosolization chamber to airways of the patient. The amount of powder metered and emitted as an aerosol is consistent across multiple of the consecutive cycles. At least one jet of the one or more air jets is positioned to not impinge on the powder in the metering space.

An air-jet DPI may include small diameter flow pathways for the inlets and outlets connected by an aerosolization chamber. The air-jet DPI is responsible for aerosolization of the powder as well as holding the full dose of powder using separate chambers. As the air source is actuated, high speed jets of air pass through the aerosolization chamber and either around or through the preloaded powder. The one or more inlets and the air jets they produce are arranged to facilitate powder aerosolization. Air jets contribute to passive cyclic loading insofar as they remove one load of powder so that further powder may enter and accumulate in a powder bed within the aerosolization chamber after the air jet is gone. In some embodiments, the air jets themselves may also contribute to a passive cyclic loading action which is concurrent with the present of the air jets.

Elements of some embodiments may include small diameter inlet flow passage(s), an aerosolization chamber, a powder reservoir (where at least a full dose of powder is initially loaded), and outlet capillary.

A powder reservoir may be positioned above (with respect to gravity) the aerosolization chamber. A powder reservoir may be centered perpendicular to the direction of primary air flow. The powder reservoir may be filled with the desired full dose (mass) of powder and then connected to the air-jet DPI with, for example, a twist-lock and O-ring seal. An exemplary powder reservoir intended for use with infants may accommodate a powder volume up to 0.55 mL or 1.5 mL, for example.

The configuration of inlet(s), aerosolization chamber geometry, and outlet(s) may differ among alternative embodiments.

An exemplary DPI may have one (e.g., a single) or multiple outlets from the aerosolization chamber. Each outlet may be or include an outlet capillary. An outlet capillary, if employed, may have a flush or protruding configuration. In a flush configuration, the capillary does not protrude into the aerosolization chamber. In a protruding configuration, the capillary tube protrudes into the aerosolization chamber by a small distance, e.g., 0.5 mm.

An exemplary DPI may have one (e.g., a single) or multiple (air) inlets into the aerosolization chamber. Inlets may be formed into the structure of the air-jet device.

It is desirable in some embodiments that at least one inlet of the one or more inlets is positioned so that an initial air jet from the at least one inlet does not enter the space occupied by loaded powder preceding actuation. Relatedly, it is desirable in some embodiments that at least one inlet of the one or more inlets is positioned so that an initial air jet from the at least one inlet does enter the space occupied by loaded powder preceding actuation. Thus, for example, an embodiment may have multiple inlets all directing the inlet airflow around the initial powder bed. Conversely, an embodiment may have multiple inlets with one directing a portion of the inlet flow toward the powder bed and the others directing the airflow through some other region of the aerosolization chamber.

With a passive cyclic loading device, introduction of a metering element, such as a powder tray, may be used to further control the amount of powder that is aerosolized with each actuation. Advantages of the passive cyclic loading system include metering a consistent amount of powder for each actuation while protecting the powder in the reservoir from aggregate formation. Aggregate formation can undesirably reduce aerosolization performance (making a larger aerosol) or reduce emitted dose from the device. A single device is able to accommodate a variable range of powder doses and aerosolize a relatively consistent amount of dose with each actuation, while remaining insensitive to the amount of powder loaded (above a certain threshold). A metering system may control the dose loaded into the aerosolization chamber that is then converted to an aerosol with each actuation.

Passive cyclic loading allows for a highly efficient, rapid, and non-invasive form of aerosol delivery to subjects from neonates to adults with selectable dosage and expected consistent performance. The amount of powder metered each cycle is consistent across multiple or consecutive cycles. It should be appreciated that “multiple” may but does not necessarily mean “all” cycles. In general, some embodiments may include a metering system which administers a consistent dose per actuation after reaching a steady state condition. The first cycle (or even first few cycles) may be generally less consistent than subsequent cycles. In addition, the final cycle may be generally less consistent than preceding cycles. The latter is especially to be expected when the total dose is not a perfect multiple of the generally consistent metered dose per cycle. For illustration, if a total dose of 22 mg of powder is prescribed for delivery to a patient, but the DPI is configured to deliver consistent doses of 5 mg of the powder for multiple cycles, 5 cycles of loading then actuation are expected to be needed. Because 22 does not divide evenly into 5 cycles, the final cycle is reasonably expected to have a unique dose inconsistent with the other cycles. The first four cycles may each deliver approximately 5 mg of powder, but the final cycle delivers approximately 2 mg of powder since this is all that would remain available in the DPI (reservoir included).

The reservoir in some DPIs may be removable. A reservoir containing powder may be inserted into a platform (e.g., connected with an aerosolization chamber) for dosing. After sufficient cycles have been administered, the reservoir may be removed, even if it still contains a remaining amount of powder. The removed reservoir may be resealed and reused. This is advantageous in applications such as multi-dose/multi-patient dosing. Alternatively, the reservoir may be a single use dose containment unit, that stores the dose until use and then is disposed of after use.

A single “cycle” of use of a DPI may generally include a step of loading the aerosolization chamber and a step of actuation which aerosolizes the loaded dose and delivers it to the subject. An exemplary loading step may be passive, e.g., the loading occurs substantially all from the influence of gravity alone. By contrast, an “active” loading may require, for example, a motor or human hand physically moving a component to cause loading (e.g., pushing a button or turning a dial). An electromechanical or physical means (vibrational or valve opening) may be used to empty (or contribute to emptying) one dose of powder into an aerosolization chamber (and, for some embodiments, onto a metering shelf in the aerosolization chamber). An exemplary actuation/aerosolization step may be passive or active. A passive actuation may be, for example, a user inhaling without any assistance. The force of inhalation is the sole force which causes air flow and resulting aerosolization. An active actuation, by contrast, may involve a motor or person physically pushing air toward the patient (e.g., with a syringe or bellow or the like). While not the only arrangement contemplated by this disclosure, many exemplary embodiments combine a passive loading with an active actuation/acrosolization. Alternative embodiments may employ some combination of active and/or passive loading together with active and/or passive actuation/aerosolization.

Medications desirable for inhalation as a powder may include, but are not limited to, antibiotics, antivirals, surfactants, steroidal or non-steroidal anti-inflammatories, clearance enhancers/agents, therapeutics for pulmonary artery hypertension, gene therapies, monoclonal antibodies, bronchodilators and corticosteroids for asthma management, growth hormone, osmotic agents, mucolytics, and combinations thereof. Some embodiments may entail delivery of systemic mediations without needles such as but not limited to: insulin, human growth hormone, rapid treatments for stroke, Alzheimer's therapies, and “nose-to-lung delivery”. Some exemplary embodiments may deliver vaccines for respiratory viruses such as but not limited to influenza, COVID-19, and similar viruses.

Various embodiments may include one or more of the following features: continuously curving walls and no sharp corners inside the aerosolization chamber; sections of the aerosolization chamber interior existing above and below the powder support surface; multiple inlets on one side of the aerosolization chamber and one or more outlets on the opposite side of the aerosolization chamber; when using multiple inlets, one or two air-jets arranged to impinge on the powder bed.

In some embodiments with multiple inlets, at most one of the air jets or at most 30% or at most 35% of the inlet flow impinges on the initial powder bed. In some embodiments at most two of the air jets or at most 70% of the inlet flow impinges on the initial powder bed.

Some embodiments may include one or more mesh structures or other porous structures. For instance, some embodiments may include a mesh or rod array in a patient interface. In addition or in the alternative, some embodiments may include a mesh or other porous structure on one or both sides of the metering shelf. Meshes or other porous structures may be used to improve deaggregation. When used at either end of the metering shelf, the mesh or other porous structure may improve metering and assist in retaining the powder on the shelf during and after loading but prior to actuation. This may be particularly helpful to allow the device to be held on an angle. During actuation the air can pass through the mesh or other porous structure to still entrain and carry off the powder as an aerosol.

An exemplary DPI may be an inline DPI in an aerosol delivery platform used for trans-nasal aerosol delivery or nose-to-lung aerosol delivery, for example.

Geometry of the aerosolization chamber is configured to efficiently form a small particle aerosol and provide a high emitted dose (ED).

Some exemplary embodiments advantageously achieve efficient and consistent lung delivery of aerosols. At a minimum it is desirable that ~45% or more of loaded dose reaches the intended part(s) of the respiratory system. To assist in achieving this performance criteria, exemplary embodiments may have a metering system which limits dose per actuation to, for example, less than 10 mg powder, or less than 9 mg powder, or less than 8 mg powder, or less than 7 mg powder, or less than 6 mg powder, or less than 5 mg powder. The upper threshold depends on the patient, especially the patient's age (e.g., for most medicaments an infant will require a smaller upper threshold to dose per actuation than a child, and a child will require a smaller upper threshold to dose per actuation than an adult). Limiting dose per actuation to an amount less than the total dose to be administered prevents overly rapid aerosol administration which in turn can lead to poor delivery efficiency to the respiratory system. Advantageously, the metering system may be configured to achieve an upper limit to dose per actuation which is generally independent of actuation volume or actuation rate.

Total dose for a single sitting may be, for example, in the range of 10 mg to 150 mg powder. Some osmotic agent, surfactant or other applications may require doses in the range of 200, or 300 or 400 mg of powder. Lung delivery performance may be kept relatively unchanged up to dose loadings of, e.g., 150 mg at a powder bulk density of 0.1 g/cm3. In general, the number of cycles required may be varied to achieve a target dose per actuation as well as target total dose. As an illustrative calculation, a total dose of 30 mg may be delivered in an exemplary method with total dose per actuation of approximate 5 mg or less. Accordingly six cycles (30 divided by 5) would be prescribed. An extra cycle or two may be used as a margin of safety for complete emptying of the aerosolization chamber.

In some embodiments, the opening which admits powder into the aerosolization chamber (or a powder delivery tube if present) may be fitted with an adjustable shutter which permits a user to manually change the degree of constriction (adjust between smaller and larger diameters and/or adjust the position of the upper edge of the opening) to tune the loaded dose per actuation.

Inlet orifices may be round or rounded. Outlet orifices may be round or rounded. The walls which enclose an aerosolization chamber may be continuously curving with no flat surfaces or sharp corners with the exception of the orifice openings. A section of the aerosolization chamber may exist on either side of the inlet and outlet orifices. An inlet orifice flow pathway on one side of the aerosolization chamber may be directly aligned with the outlet orifice flow pathway on the other side of the aerosolization chamber. The inlet orifice may have a smaller diameter than the outlet orifice. If multiple inlet orifices and a single outlet orifice, the sum the inlet orifice areas may be less than the area of the outlet orifice. An inlet orifice diameter may be less than a minimum characteristic length or diameter of the aerosolization chamber divided by a factor of 1.5. The vertical linear section above the inlet orifice may be at least 10% of the total linear vertical distance in the orientation of intended use. The vertical linear section below the inlet orifice may be at least 50% of the total linear vertical distance in the orientation of intended use.

An aerosolization chamber for forming an aerosol may comprise multiple rounded inlet orifices on one side of the chamber all predominately directing initial air-jet flows toward one or more outlet orifices on an opposite side of the aerosolization chamber. The enclosing walls of the aerosolization chamber may be continuously curving with no flat surfaces or sharp corners with the exception of the orifice openings and opening for powder loading.

An exemplary aerosolization chamber may be configured to hold between 1 and 100 mg of dry powder. The total initial dose contained in the powder reservoir may be between 10 and 400 mg and would vary depending on intended dose and subject size. For test animal and veterinary applications, these ranges may be exceeded.

An exemplary aerosolization chamber may be operated with a positive pressure gas source with a device pressure drop in the range of 0.5 to 8 kPa, or in the range 0.5 to 6 kPa. Alternatively, an exemplary aerosolization chamber may be operated with negative pressure generated from a subject's inhalation with a device pressure drop in the range of 0.5 to 4 kPa.

In some embodiments device actuation may be timed with infant inhalation and a safety pressure (pop-off) valve may be included which limits pressure exposure for any mistimed device actuations.

In some embodiments a mass flow meter may be connected between the air source and air-jet DPI. The mass flow meter may be used to calibrate and verify the air source actuation parameters.

Inlets and outlets to an aerosolization chamber may provide a continuous flow pathway and means for the powder to exit the interior space of an aerosolization chamber without physically piercing, crushing, puncturing, crushing, rupturing, or cutting a containing wall of the aerosolization chamber. Alternatively, inlets and outlets may be created just before use of the aerosolization chamber by an action such as but not limited to puncturing or piercing.

The configuration (e.g., size, shape, arrangement with respect to the interior space(s) of the aerosolization chamber, and arrangement with respect to other parts of the aerosolization chamber such a shelf if present) of the inlet(s) and outlet(s) assists in providing the hydrodynamic force needed to deaggregate powder. The hydrodynamic force may take the form of one or more inlet jets and secondary airflows.

An inlet's direct airflow path within an aerosolization chamber may be defined as a path of airflow aligned with the inlet (e.g., coaxial with inlet center axis) which, due to momentum, continues on a straight line until it reaches a boundary or outlet. At least a portion of the direct airflow path may be characterized as an “inlet jet” or similar. Any remaining portion of the path is not an inlet jet. A Reynolds number (Re) threshold may be used to define a boundary in space up to which an inlet jet exists and beyond which the inlet jet no longer exists. For a jet to occur and move through a majority of space of an aerosolization chamber, for jets not impinging on a wall boundary, the Re may preferably be 100 or greater. For Re<100, the jet may only move through a portion of the aerosolization chamber. The inlet jet may be assumed to end at a location in which the secondary velocities (reverse flow) on a plane normal to the inlet jet are greater than the velocity of the inlet jet. Reynolds number may be determined according to the following equations:

$$Re ≡ \frac{inertial forces}{viscous forces}$$

$$Re = \frac{ρ {V}_{jet} {D}_{jet}}{μ}$$

where
ρ=air density 1.17 kg/m³,
Vⱼₑₜ≡inlet jet velocity,
Dⱼₑₜ ≡inlet orifice diameter,
μ≡dynamic viscosity of air=183.7×10⁻⁷ (N·s/m²).
   Note that Re is a non-dimensional parameter.

Some aerosolization chambers may be configured such that inlet flow forms an air jet aligned with the inlet orifice. For instance, the inlet jet's center axis may be coaxial with an inlet's center axis. Unless the jet impinges on a wall boundary, an inlet jet may traverse a majority of the aerosolization chamber based on the jet's momentum (e.g., travel at least 50% of the distance between an inlet orifice and an outlet orifice or containment wall directly opposite the inlet orifice). An inlet jet may traverse at least 50%, at least 60%, at least 70%, at least 80%, at least 90%, or 100% of the distance between an inlet orifice and an outlet orifice or containment wall directly opposite in the inlet orifice. An inlet jet may traverse no more than 60%, no more than 70%, no more than 80%, no more than 90%, or no more than 99% of the distance between the inlet orifice and an outlet orifice or containment unit wall directly opposite the inlet orifice. The exact configuration desired for a particular aerosolization chamber depends on various aspects such as the relative positions of inlet/outlet orifices and the location of the powder bed. Regardless of the configuration of the inlet jet, in many embodiments it is desired that at least one inlet jet at no point make contact with the powder bed. More generally, in some embodiments it may be preferable that the powder is not in the direct path of at least one inlet air jet (e.g., a linear path corresponding with the flow path of inlet jet but which may extend past the end of the inlet jet).

Those of ordinary skill in the art will recognize that the structures of exemplary embodiments disclosed herein may be made of suitable materials known in the art such as certain plastics, metals, and the like. For instance, inlet and outlet orifice flow passages may be constructed with hollow metal capillaries and are often referred to as inlet and outlet capillaries, whether or not the passages are made of metal or another suitable material.

For devices intended for use with infants, the total air space volume (the dead space) of the air jet DPI system may be 5 ml or less (excluding the volume of the air source, which can be variable). A total air space volume of the air jet DPI system may be 2 ml or less. The total air space volume may be determined from air jet inlet orifice (which may be a capillary) through to the end of the patient interface, so parts would include the aerosolization chamber, outlet capillary, and patient interface. For devices used with children or adults, the total air space volume is less of a concern and may be considerably larger for larger air actuation volumes.

An exemplary infant air-jet DPI system may deliver both the aerosol and up to a full inhalation breath to the infant in a short amount of time (e.g., <1 sec for inhalation) and can be used to maintain a short breath hold. The use of positive pressure to deliver the aerosol and inhalation breath expands the flexible upper airways and may enable deeper than tidal volume inhalation and improved lung penetration of the aerosol. As with manual ventilation with a bag and mask interface, this approach may also help to open closed or obstructed lung regions, further increasing the reach of the inhaled aerosol.

Delivered gas to form the aerosol and support infant respiration depends on infant weight with a typical range of 6-8 ml of gas per kg of infant body weight (i.e., 6-8 ml/kg). For a preterm infant weighing 1600 g, potential delivered gas volumes may range from 10 to 13 ml with a preferred value of approximately 10 ml. For a full-term infant weighing 3550 g, potential delivered gas volumes may range from 21 to 28 ml. It is desirable for an administering physician to have precise control over the amount of air delivered to an infant and to be able to adjust this air volume depending on any lung injury or specific case being treated. The lower end of delivered gas volume to extremely preterm infants may be 4-5 ml, for example, and the upper end to much older infants suffering acute respiratory distress may be as high as 100 to 200 ml. For aerosol delivery to test animals, the delivered gas volume range may be extended to ~1 mL to >200 mL. Similarly, for adults and children, the delivered gas volume range may be as low as 4-5 ml, when the device injects aerosol into a partial breath, or as high as 1.5-4 L, when the device provides a full inhalation breath.

Inlets and outlets may be oriented along the long/longitudinal axis of the aerosolization chamber or at a non-zero angle with the longitudinal axis, e.g., perpendicular.

According to aspects of some exemplary air jet DPIs, the air jet axis is perpendicular to the longitudinal axis of the aerosolization chamber. The longitudinal axis of the aerosolization chamber has a vertical orientation in a state of use. At least one of the one or more inlets is aligned on a common axis with at least one of the one or more outlets. The air jet axis passes only through an upper longitudinal segment of the aerosolization chamber. The one or more inlets and the one or more outlets are all positioned at an upper longitudinal segment of the aerosolization chamber. The upper longitudinal segment may extend no more than 50% of a length (or 25% of the length) of the aerosolization chamber. A lower longitudinal segment of the aerosolization chamber is removable and reattachable to the upper longitudinal segment. The lower longitudinal segment is opposite the upper longitudinal segment.

A bottom portion or segment of an aerosolization chamber may unscrew from a complementary top portion via a reversable attachment mechanism such as threaded screws and/or one or more magnets. The attachment mechanism may include one or more silicone o-rings to provide a seal between the bottom and top portions of the aerosolization chamber.

An aerosolization chamber may be configured consistent with the volume and shape of a Size 0 capsule, though the volume and shape may vary among embodiments to accommodate other volumes.

### BRIEF DESCRIPTION OF THE DRAWINGS

FIG. 1 is a block diagram of an exemplary air-jet dry powder inhaler (DPI) platform.

FIGS. 2A-2D are an exemplary dry powder inhaler (DPI).

FIGS. 3A and 3B are the DPI of FIGS. 2A-2D shown loaded with powder.

FIGS. 4A-4D are a visualization of the metering space in the DPI of FIGS. 2A-2D.

FIGS. 5A-5D are another exemplary DPI.

FIG. 6A is part of yet another exemplary DPI.

FIG. 6B is an assembly of inhaler components which include the DPI of which FIG. 6A is a part.

FIGS. 6C and 6D are yet another exemplary DPI.

FIGS. 7A, 7B, and 7C are exemplary inlet and outlet arrangements for aerosolization chambers of DPIs.

FIG. 7D is a DPI with a single inlet, single outlet configuration.

FIG. 7E is a DPI with a triple inlet, single outlet configuration.

FIG. 8A is an assembly which includes a fixed volume powder reservoir.

FIG. 8B is an assembly which includes a variable volume powder reservoir.

FIGS. 9A-9D are alternative configurations compared in the Examples.

FIG. 10A shows device emptying characteristics from Example 7 for 10 mg total dose.

FIG. 10B shows device emptying characteristics from Example 7 for 30 mg total dose.

### DETAILED DESCRIPTION

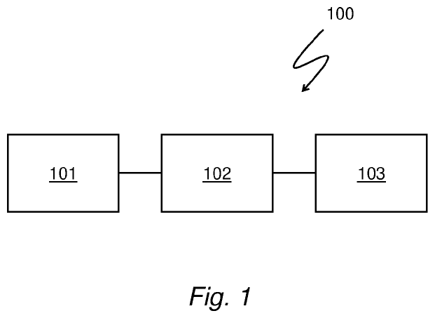

FIG. 1 is a block diagram of air-jet dry powder inhaler (DPI) platform 100 for rapid and efficient aerosol administration, e.g., by nose-to-lung administration. Overall, the platform 100 may be configured to provide a full inhalation breath along with the aerosol to the lungs of a patient, e.g. an infant, through the nasal route. The modality of administration may be referred to as a direct-to-infant approach in this case.

The platform 100 comprises an air source 101, an air-jet DPI 102, and a patient interface 103. The air source 101 provides aerosolization energy and inhalation gas. The air source is responsible for providing the aerosolization energy to the air-jet DPI 102 as well as delivering the aerosolized powder to the lungs while providing a full or partial inhalation breath for the patient. An exemplary air source 101 at baseline conditions may deliver for an infant patient an air actuation volume (AAV) of 10 mL, for example, delivered in bursts at a Q90 flow rate of 1.7 L/min. A variety of exemplary air sources suitable for use with infants are disclosed in U.S. patent application Ser. No. 17/794,875 filed Jul. 22, 2022 (published as US20230071308A1), which is herein incorporated by reference. Exemplary infant AAVs include 5-30 mL. Exemplary adult AAVs include 5 mL-1.5 L, or more, depending on the patient. Exemplary flow rates for infants include 1.5-6 L/min. Exemplary flow rates for adults include 1.5-30 L/min or up to 45 L/min. These numbers are generally applicable to human subjects. It should be appreciated that exemplary devices and methods herein may, if desired, be employed for patients/subjects that are animals such as but not limited to mice, rats, dogs, cats, horses, livestock, and exotic animals.

The air-jet DPI 102 is configured for holding the loaded powder and aerosolizing metered doses per actuation. After exiting the air-jet DPI 102, the formed aerosol then passes through the interface 103. The patient interface 103 transports the aerosol from the DPI to the patient. An exemplary patient interface is a nasal interface which effectively delivers the aerosol from the DPI to the patient's nasal passage. An exemplary nasal interface may form an airtight steal with the patient's nostrils. The prong of the nasal interface may be inserted approximately 5 mm into the nose, for example, consistent with short ventilation support nasal prongs. In some embodiments a nasal interface may comprise a mesh (e.g., a metal mesh) to assist in reducing NT deposition.

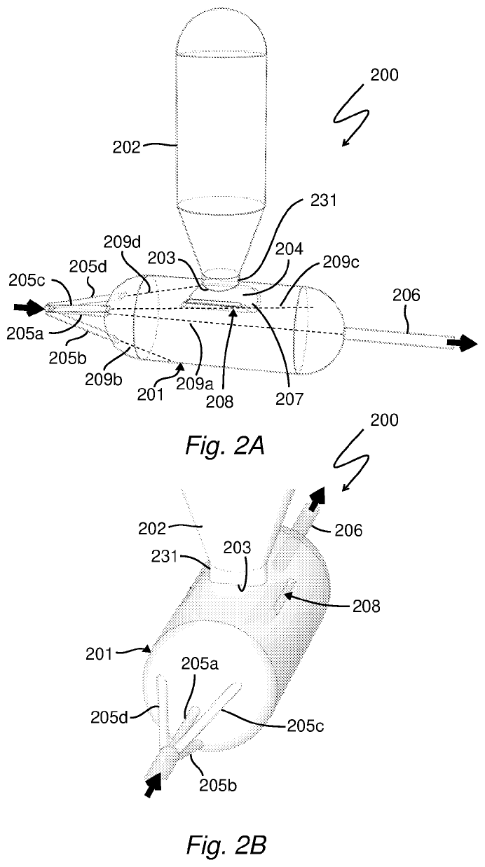

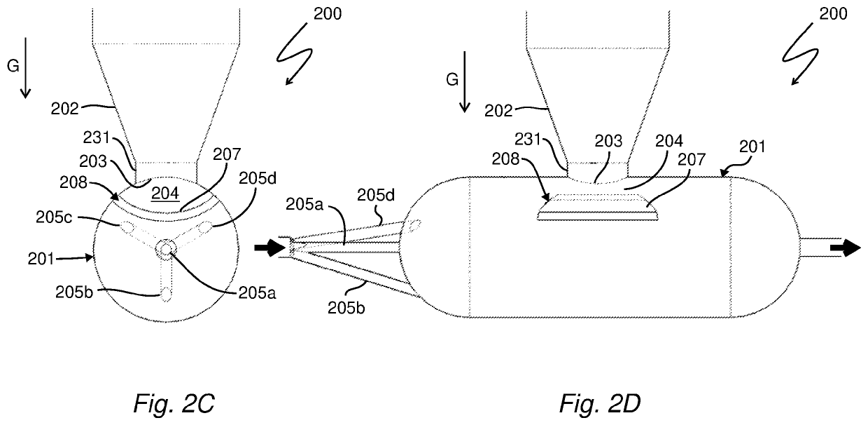

FIGS. 2A, 2B, 2C, and 2D are alternative views of an exemplary dry powder inhaler (DPI) 200. The DPI 200 includes an aerosolization chamber 201, a reservoir 202, one or more air inlets 205, and one or more aerosol outlets 206. The reservoir 202 is configured to hold at least a full dose of powder to be delivered to a patient. An opening 203 is configured to permit powder from the reservoir 202 to fall into the aerosolization chamber 201 from gravity alone. The aerosolization chamber 201 includes a powder support surface 207 on which powder which falls through opening 203 accumulates into a powder bed. The reservoir 202 is positioned, arranged, and/or attached to the aerosolization chamber 201.

A metering space 204 exists between the powder support surface 207 and opening 207. The metering space 204 is configured to fill with powder of an amount less than the full dose when the reservoir 202 is positioned above the powder support surface 207 with respect to gravity.

The aerosolization chamber 201 has a space substantially enclosed by enclosing walls and a plurality of air inlets 205 (205a, 205b, 205c, and 205d). The one or more air inlets 205 to the aerosolization chamber 201 are each configured to produce an air jet 209 in the aerosolization chamber 201. The dotted lines in the figures trace the center line of each air jet 209a, 209b, 209c, and 209d. At least one inlet of the one or more inlets 205 is positioned so that an air jet from the at least one inlet does not enter the metering space 204. As illustrated in FIG. 2A, the inlets are configured such that the four inlet air jets 209 entering the aerosolization chamber 201 do not (and cannot) directly impinge upon powder resting on the powder support surface 207. The air jets 209c and 209d are of a height with the powder support surface 207, but the inlets 205c and 205d are positioned so that the air jets 209c and 209d pass to either side of the powder support surface 207 instead of passing over the powder support surface 207. Alternatively, for some embodiments, at least one inlet of the one or more inlets 205 may be positioned so that at least one air jet 209 from the at least one inlet does enter the metering space 204. As a result, the air jet would impinge on a powder bed resting on top of the powder support surface 207.

The aerosolization chamber 201 is responsible for aerosolization of the powder. As the air source is actuated, high speed jets of air pass through the aerosolization chamber 201 and either around or through the preloaded powder, facilitating powder aerosolization. Air jets 209 constitute primary air flows. Secondary air flows may develop as the air jets 209 strike walls and induce secondary flows through aerodynamic effects. Generally, the aerosolization chamber 201 may be scaled against air flows in and out of the chamber's interior during use except for those from inlets 205 and outlet 206. In addition, an amount of air may enter or exit the aerosolization chamber in connection with the volume of air space displaced by powder entering the chamber. This occurs when the device is between actuations and the speeds associated with this movement of air are negligible. Accordingly this movement of air has no significant impact on aerosolization. During an actuation of the DPI, clean air may be admitted to the aerosolization chamber through inlets 205. Air with entrained powder exits the aerosolization chamber through outlet 206. In a similar embodiment, a negative inhalation pressure arising from the subject pulls air through the air inlets, aerosolization chamber, and outlet supplementing or taking the place of the positive pressure air source, but generating a near identical airflow pattern within the device.

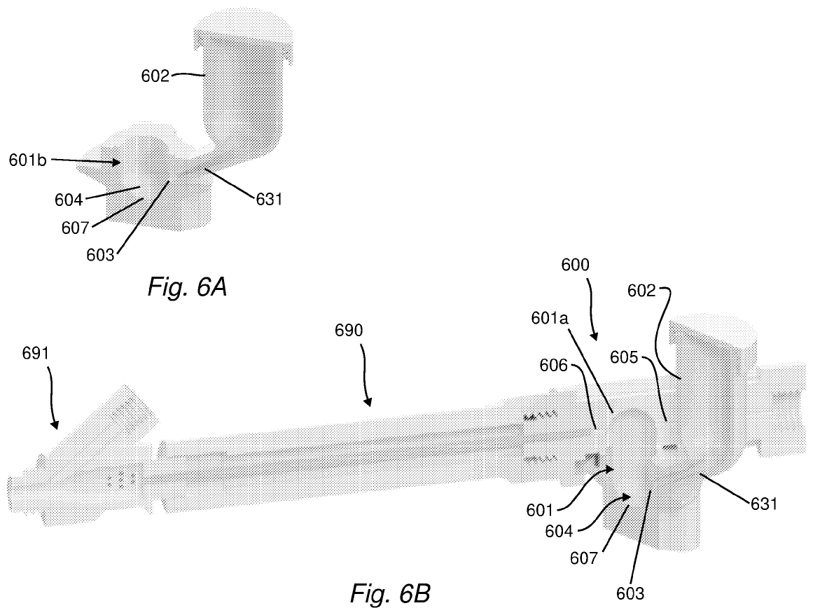

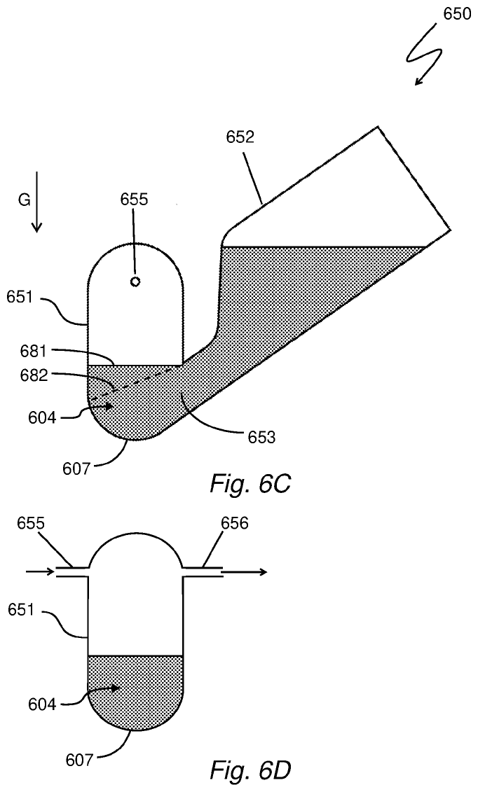

The reservoir 202 is configured to hold the full dose of powder to be administered to a patient in a single treatment. The full dose may be referred to as the total dose. The reservoir 202 is positioned above (with respect to gravity) the aerosolization chamber 201. More specifically, the opening 203 through which powder exits the reservoir 202 is positioned above the powder support surface 207 with respect to gravity. In this disclosure, when a first object is above a second object with respect to gravity, it means the first object is more distant from Earth than the second object. Another way of saying this is that the first object has more gravitational potential energy than the second object. A first object above a second object with respect to gravity does not necessarily mean the first object is directly above the second object such that lowering the first object or raising the second object would cause the two objects to collide. This arrangement is possible but not requisite when the first object is above the second object with respect to gravity. In the illustrated embodiment of FIGS. 2A-2D, the reservoir 202 is both (i) positioned above the powder support surface 207 with respect to gravity and (ii) directly above the gravity support surface 207. The same is true of the relationship between opening 203 and powder support surface 207. An alternative arrangement is illustrated by FIGS. 6A-6D which will be discussed below.

Reservoir 202 may be centered perpendicular to the direction of primary air flow in the aerosolization chamber 201. Alternative embodiments may not have this particular arrangement. Generally, the direction of primary airflow may be the shortest geometric line from the inlets to the outlet. The average vector of the inlets, if there are multiple inlets, may be the primary airflow direction. In DPI 200, the primary airflow direction corresponds with air jet 209a.

Note that the view in FIG. 2C omits illustration of the outlet 206. From the perspective of FIG. 2C, the outlet 206 coincides with the position of inlet 205a. In FIG. 2D, inlet 205c is omitted from illustration since it would obscure the view of inlet 205d according to the perspective of FIG. 2D.

Reservoir 202 may be filled with the desired dose (mass of powder) and then connected to the aerosolization chamber with an airtight connection. An exemplary airtight connection is a twist-lock and O-ring seal configuration. Reservoirs may be sealed after filling with, e.g., foil covers or screw caps.

The volume of the reservoir may vary among embodiments depending on the intended use. For example, a reservoir intended for holding a full dose for treatment of an adult patient may generally be larger than a reservoir intended for holding a full dose for treatment of an infant, assuming the same powder formulation is intended for both the adult and infant. As a non-limiting example, an exemplary powder reservoir may be configured to accommodate a powder volume up to 0.55 mL. This size accommodates dose loadings between 10-50 mg of the AS-EEG formulation presented in the Examples below which had a bulk density of ~0.1 g/cm³. A larger size up to 1.5 mL for example may accommodate up to approximately 150 mg of the same powder. Density varies among medicament powders, so the exact maximum loaded powder mass which will fit in a given reservoir volume varies to some degree depending on the formulation. Reservoir shape may vary among embodiments as well. One non-limiting example is a half capsule shape. In some embodiments the reservoir may include a capsule or receptable for receiving a capsule containing a dose of powder. One standard size for existing capsules is 7.1 mm diameter.

Exemplary DPIs include a metering system which includes one or more metering elements. In the context of DPI 200, the opening 203, metering space 204, and powder support surface 207 are central features of the metering system. For embodiments which have a powder delivery tube 231 (PDT), this feature may or may not constitute part of the metering system. Generally, the metering system controls the amount of powder (volume and/or mass) which is subjected to primary and/or secondary flows in the aerosolization chamber. Powder must physically enter the aerosolization chamber to be subject to significant primary and/or secondary flows. It is advantageous for the opening 203 to be arranged in a position and sized so that no significant flows in the aerosolization chamber pass through the opening 203 and out of the aerosolization chamber 201.

The DPI 200 is configured to have a passive cyclic loading action. The total dose of powder a patient is to receive in a single sitting is divided by the metering system across multiple inhalations. The metering system controls the dose of powder loaded into the aerosolization chamber that is then converted to an aerosol with a single actuation. The loading action is a gravity fed action. Generally speaking, gravity is the only force employed to move powder from the reservoir into the aerosolization chamber. It is possible that during actuation the flows in the aerosolization chamber may play a minor role in further powder descending into the aerosolization chamber through opening 203. However, as already stated, the opening 203 is configured so that no primary flows and a bare minimum of (preferably zero) secondary flows pass through opening 203, making any contribution of flows to reloading of the aerosolization chamber negligible.

Benefits of the passive cyclic loading system include metering a consistent amount of powder for each actuation while protecting the powder in the reservoir from aggregate formation. For some embodiments, any two cycles (each entailing a load step then actuation step) being compared may be regarded as having consistent loads with one another if either cycle's powder load (which is then emitted) is within +/−30% of the other cycle's powder load (which is then emitted) by volume. For some embodiments, the consistency may be within +/−10% of one another's powder load (which is then emitted) volume. For some embodiments, any two load cycles being compared may be regarded as having consistent loads (which are then emitted) with one another if either cycle's powder load (which is then emitted) is within +/−30% of the other cycle's powder load (which is then emitted) by mass. For some embodiments, the consistency may be within +/−10% of one another's load (and then emitted) mass. This consistent amount of metered powder would occur outside of possible start-up and trail-off effects. It will be understood that a small difference may exist between how much powder (e.g., the mass) descends into the aerosolization chamber on a given cycle versus how much aerosol (e.g., the mass) leaves the aerosolization chamber on the same cycle. Generally the explanations of this disclosure treat these two amounts as typically equal to one another within a margin of error. For this disclosure, it can be reasonably assumed that if two loads are consistent with one another, the corresponding masses of emitted aerosol are consistent with one another.

The metering space 204 is the physical volume within the aerosolization chamber which fills with powder solely under the influence of gravity and assuming sufficient powder is available in the reservoir to transfer into the metering space 204. The size of the metering space is controllable by a combination of the size and shape of the powder support surface 207 and its spatial relationship with opening 203. Generally speaking, the elements are arranged such that powder will continue to fall into the aerosolization chamber under the influence of gravity so long as and until there is no more free space available for powder to move into directly below the opening 203. The powder support surface 207 physically prevents the accumulating powder from falling any lower. Powder support surfaces may be different shapes or contours in different embodiments. For instance, a powder support surface may be any of various elliptical curves or flat. In DPI 200, the powder support surface is curved, arcing between two sides of the aerosolization chamber which maintain the powder support surface 207 at a fixed distance from both the floor and ceiling of the aerosolization chamber.

The powder support surface 207 may belong to a shelf 208. The powder support surface 207 may be the top surface of the shelf 208. A shelf may also be referred to as a tray in this disclosure. The shelf 208 is a metering element which may be used to help control the amount of powder that is aerosolized with each actuation.

In DPI 200 the powder reservoir 202 is located above a powder shelf 208 in the aerosolization chamber 201 as a means to control (or contribute to the control of) the dose loaded into the aerosolization chamber 201 that subsequently forms an aerosol with each actuation. The position of the shelf 208 and volume of the shelf 208 are two aspects of the shelf 208 which may be adjusted depending on the embodiment to influence the volume of the metering space and accordingly the volume of metered powder when the DPI 200 is in use. In addition, the geometric shape and size (e.g., circular and diameter) of the opening 203 connecting the powder reservoir 202 with the metering space 204 above the powder shelf 208 may be selected based on the desired volume of the metering space 204. The diameter of the opening is sufficiently large to promote free motion of the powder onto the shelf under the influence of gravity when the metering space is not yet filled. Conversely, the diameter of the opening is sufficiently small to protect powder in the reservoir from exposure to repeated aerosolization forces.

Metering system features may be adjusted among embodiments for controlling the amount of powder transferring from the powder reservoir to the aerosolization chamber during each actuation. The powder metering system may be tuned for individual powder formulations to maintain high efficiency lung delivery via the nose-to-lung route over multiple device actuations.

An “inlet” is one or more structural elements which, at a minimum, define an orifice through which matter (e.g., a gas) may flow. Similarly, an “outlet” is one or more structural elements which, at a minimum, define an orifice through which matter (e.g., gas and entrained powder particles) may flow. An inlet may further comprise a protrusion that extends inwardly or outwardly. An outlet may further comprise a protrusion that extends inwardly or outwardly. Whether in regard to an inlet or an outlet, an inward protrusion extends from a surface (e.g., of a containing wall) toward or partially toward the aerosolization chamber's center. An outward protrusion extends from a surface (e.g., of a containing wall) away or partially away from the aerosolization chamber's center. Protrusions may be of circular cross-section, and some protrusions may be referred to as capillaries. Protrusions may have cross-sectional shapes other than circular, e.g., oval, oblong, square, rectangular, polygonal, or some other shape.

When a high velocity jet of air enters the aerosolization chamber, it expands creating secondary velocities. At least one inlet may be aligned with at least one outlet (coaxial center axes). Sizes of inlets and outlets (e.g., the internal diameter of an inlet or outlet with a circular cross section) may fall into any of several ranges, depending on the embodiment. As a non-limiting example, inlets and outlets may be in the range of 0.4 to 2.4 or 3 mm. A size of 3 mm or greater may be needed in certain low pressure devices. As further alternative ranges, a diameter of an inlet flow passage may be 0.3 to 1 mm; or 0.5 to 0.6 mm. As further example ranges, diameter of an outlet flow passage may be 0.5 to 1.2 mm, or 0.6 to 1.17 mm. As further possible ranges, exemplary capillary and orifice diameters may be 1.3 to 3.5 mm. These sizes may be measured at the respective orifices. The diameters may be configured to provide a controlled high speed micro jet (which may simply be referred to as a “jet” in this disclosure) at the inlet and filter large particles from exiting the outlet. These qualities help ensure production of a fine deagglomerated aerosol when the aerosolization chamber is evacuated. In some exemplary embodiments, the sizes of inlets and outlets, in particular their respective orifices, are different. An inlet orifice may be smaller than an outlet orifice, or an outlet orifice may be smaller than an inlet orifice. A larger outlet (e.g., measured by orifice diameter) relative to the inlet is advantageous in many embodiments in order to decelerate the inlet airflow and induce secondary velocities/flow in the aerosolization chamber. The secondary flows may improve dispersion and/or deaggregation of the powder bed. The sizes selected for any given embodiment may be selected based in part on the intended flow rates and pressure drops with which the device will be operated. As non-limiting examples, exemplary flow rates of air are 5 to 30 LPM for children, or in a range of 10 to 45 LPM for adults. Exemplary actuation flow volumes are 100 ml to 1.5 L or higher.

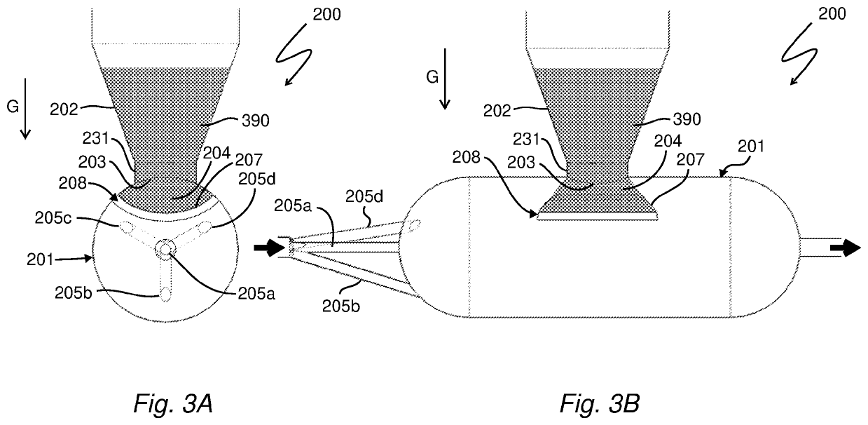

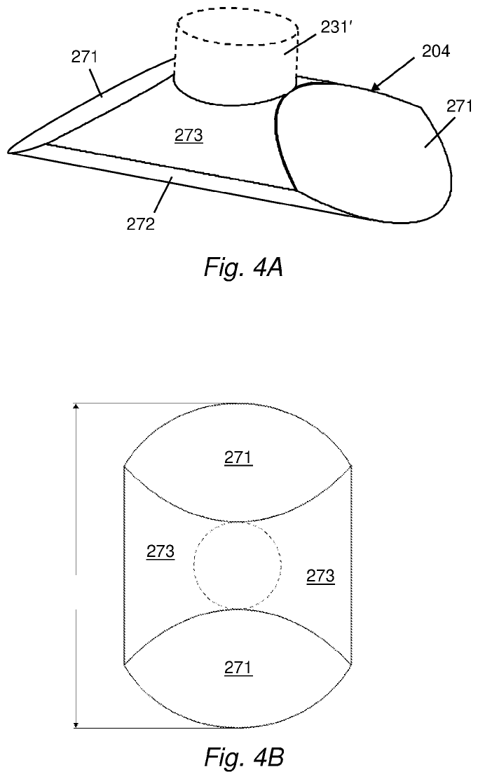

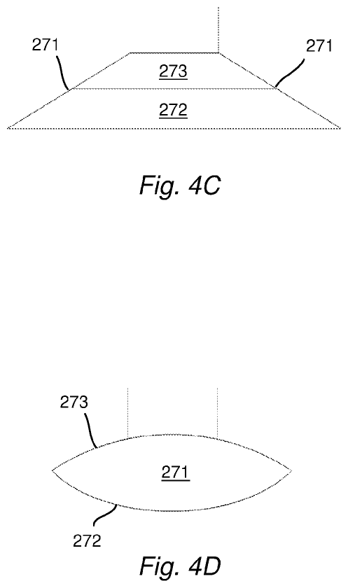

FIGS. 3A-3B depict the DPI 200 in a state of use. Powder 390 is visible both in the reservoir 200 and on the powder support surface 207. The metering space 204 is completely filled. FIGS. 4A-4D take a closer look at the metering space 204 specifically.

FIGS. 4A-4D depict the metering space 204 as a bounded three-dimensional shape to assist in visualizing its function. Volume 231′ belonging to a powder delivery tube 231 is also depicted but with broken lines. The volume 231′ is generally not a part of the metering space. When the DPI has no powder loaded, the metering space 204 contains only air. When the DPI is loaded with powder, the metering space 204 fills with powder. Actuation of the DPI substantially evacuates the metering space 204 of the powder which, prior to the actuation, was present in metering space 204. Even before the actuation is entirely complete, however, the metering space 204 may begin to refill as gravity causes powder which was still in volume 231′ of the powder delivery tube and/or reservoir to begin descending into metering space 204.

The metering space 204 in this exemplary embodiment is influenced by the size of the shelf 208. The metering space 204 has a volume created by the empty space between the surface of the inside of the aerosolization chamber and the powder support surface of the powder shelf. Owing to how a powder falls from the opening and accumulates in a pile on the shelf, the size of the shelf and distance from the opening may be selected so that the pile forms up to the edges of the shelf without significant amounts of powder falling over the edges of the shelf. In some embodiments, the shelf may be oversized so that the bottom of the powder bed does not in fact reach the edges of the shelf. Generally, a medicament powder intended for DPI applications when falling under the influence of gravity may form a generally conical pile of powder on a flat surface. Similarly, when the powder falls onto the powder support surface 207, two sides of the pile are substantially flat in cross-section. These correspond with sides 271 of the metering space 204. The remaining sides of the powder bed may conform to the sides of the aerosolization chamber and shelf. In the visualization of the metering space 204, the side 272 is formed by the powder support surface. The side 273 is formed by the roof of the aerosolization chamber.

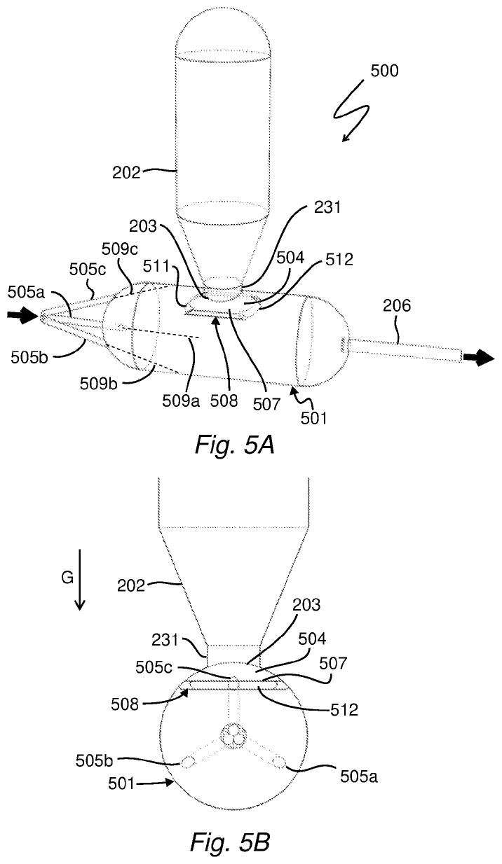

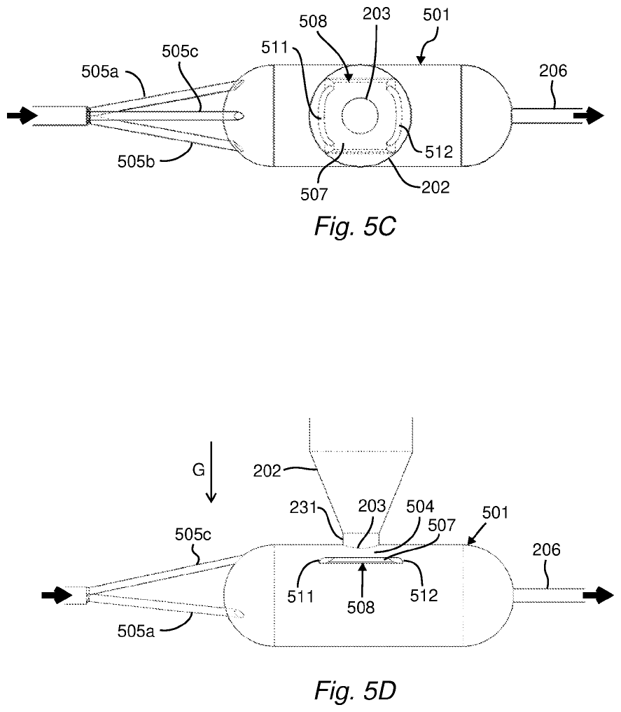

FIGS. 5A-5D depict a further exemplary DPI 500. Several features are substantively identical in both DPI 200 of FIGS. 2A-2D and DPI 500 of FIGS. 5A-5D. Such features are labeled with the same reference features. Briefly, DPI 500 includes an aerosolization chamber 501, a reservoir 202, one or more air inlets 505, and one or more aerosol outlets 206. The reservoir 202 is positioned, arranged, and/or attached to the aerosolization chamber 501.

Whereas DPI 200 had a curved powder support surface 207, DPI 500 includes a flat powder support surface 507. The powder support surface 507 is the top surface of the substantially flat shelf 508. The metering space 504 lies between the shelf 508 and opening 203. The shelf 508 has a tapered edge 511 facing in the direction of the inlets 505 and a tapered edge 512 facing in the direction of the outlet 206.

DPI 500 further contrasts with DPI 200 with respect to inlet configuration. DPI 500 has three inlets 505a, 505b, and 505c which are arranged in a symmetrical pattern with each inlet offset from a centerline of the aerosolization chamber 501. None of the corresponding inlet air jets 509a, 509b, and 509c aligns with the center axis of outlet 206. Furthermore, inlet 505c and its corresponding air jet 509c are positioned so that air from the air jet 509c enters the metering space 504. Accordingly the air jet 509c impinges on powder when the metering space 504 is filled with powder. On the other hand, both air jets 509a and 509b pass below the shelf 508 and do not impinge on the powder bed atop the powder support surface 507.

Note that the view in FIG. 5B omits illustration of the outlet 206. In FIG. 5D, inlet 505b is omitted from illustration since it would obscure the view of inlet 505a according to the perspective of FIG. 5D.

DPIs 200 and 500 above illustrate the option for a powder support surface to be embodied on a shelf which is positioned in between the ceiling and floor of the aerosolization chamber with space above and below the shelf. An alternative configuration is depicted by FIGS. 6A-6D.

FIGS. 6A and 6B depict a DPI 600 with a powder side loading configuration. FIG. 6A shows a lower aerosolization chamber section 601b which combines with an upper section 601a.

Both sections are shown assembled together to form DPI 600 in FIG. 6B, together with other exemplary platform elements. The DPI 600 is shown connected with an exemplary outlet capillary extension 690 which is in turn connected with a device 691. The outlet capillary extension 690 may be used to provide working distance between the DPI 600 and subject. It also may provide additional deaggregation. The device 691 comprises a branching port usable for releasing exhalation air flow and connection with, e.g., pressure measurement device. This may be desirable with infants, for example. With infants it is important to not over-inflate the infant lungs. This danger may be avoided by limiting air delivery to ~6-8 ml/kg, for example, and monitoring input pressure at the nasal cannula interface.

Similar to DPIs 200 and 500 discussed above, the DPI 600 has a powder reservoir 602 and opening 603 leading from the powder reservoir to the aerosolization chamber 601. Also similar to DPIs 200 and 500, the DPI 600 has the reservoir 602 and opening 603 both positioned above the powder support surface 607 with respect to gravity (the arrow labeled ‘G’ in the figures indicates gravity's direction). However, the powder support surface 607 of DPI 600 is the floor of the aerosolization chamber 601, and both reservoir 602 and opening 603 are not directly above the powder support surface 607. The reservoir 602 and opening 602 are positioned to a side of the aerosolization chamber 601. A powder delivery tube 631 angles downward and laterally from the base of the reservoir 602. Solely under the influence of gravity, powder may slide down the powder delivery tube 631 to opening 603 where it falls down a remaining vertical distance (with respect to the gravity vector) to the powder support surface 607. The powder accumulates in a powder bed on the support surface 607 until the top of the bed reaches the opening 603. Powder stops flowing into the aerosolization chamber once the powder bed pile reaches the upper edge of the opening 603. The profile shape of the reservoir, PDT, and lower aerosolization chamber section are such that the powder is able to flow freely until the edge of the powder bed nearest the opening reaches approximately the same level as the top edge of the opening, without experiencing constrictions that would limit the free flow of powder due to gravity.

FIG. 6C is a graphic of a DPI 650 similar to DPI 600. FIG. 6D shows the DPI 650 rotated such that the reservoir 652 rotates out of the illustrated plane so that both the inlet 655 and outlet 656 are visible. In DPI 650, the bottom of opening 653 smoothly transition to powder support surface 607. The DPI 650 is depicted with powder filling the metering space 604. The powder accumulated in a powder bed on the support surface 607 until the top of the bed reached the opening 653. Depending on the particular powder formulation being used, the top 681 of metering space 604 and corresponding level of the powder when the space is filled may form a substantially planar surface perpendicular to gravity's vector, as depicted by line 681, or form an angle. For example, some powders may stop filling under the influence of gravity with an angled top surface 682 or similar. The line 682 would constitute the top of the metering space in such case. Either outcome as to the shape of the metered space and powder filling that space is generally acceptable, as in either case a consistent volume would be filled after each actuation (provided sufficient remaining powder in reservoir 652). Importantly, the amount of powder which refills in either case after each actuation will remain substantially the same for the same powder.

The DPIs 600 and 650 limit the amount of powder in the aerosolization chamber at any given moment to a reproducible volume (and thus mass) of powder which is smaller than the volume of the aerosolization chamber. These DPIs advantageously eliminate the need for a metering shelf by instead using the floor of the aerosolization chamber as the powder support surface. It is advantageous in some embodiments for the powder to be admitted to the aerosolization chambers of DPIs 600 and 650 from an angle of, for example, 20-30 degrees from level. Higher angles may be desired for some embodiments, e.g., with angles above 30 degrees or above 35 degrees.

Some embodiments may have a DPI which combines a shelf for the powder support surface and a side wall position of the opening through which powder enters the aerosolization chamber.

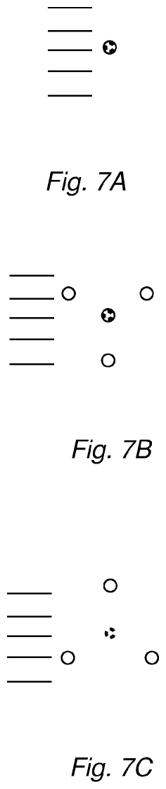

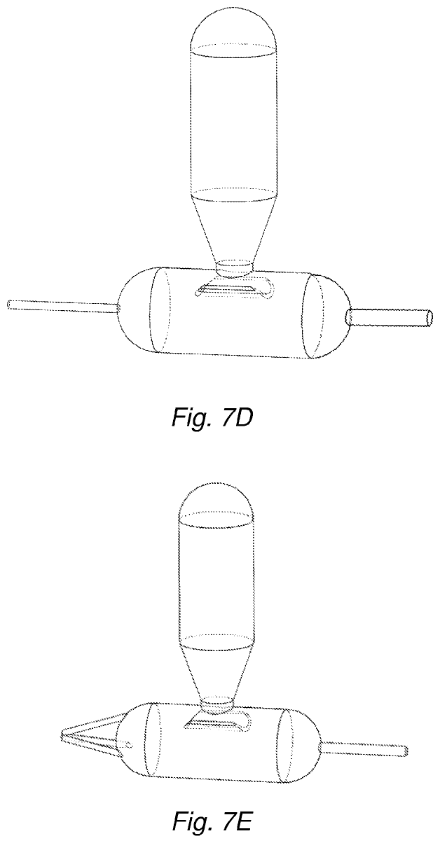

FIGS. 7A, 7B, and 7C show a non-limiting selection of alternative arrangements of inlet orifices and outlet orifices for various embodiments. In each figure the broken line circle depicts the position of an outlet orifice. The solid circles depict the relative positions of inlet orifices. The figures are oriented such that a center axis of the outlet orifice is perpendicular to the sheet. FIG. 7A depicts a configuration with a single inlet and single outlet. The center axes of both may be coaxial. FIG. 7B depicts a configuration with a single outlet and four inlets. The center axis of the outlet may be coaxial with the center axis of the center inlet. The FIG. 7B configuration corresponds with, for example, DPI 200 (see, e.g., FIG. 2C). FIG. 7C depicts a configuration with a single outlet and three inlets. The three inlets of FIG. 7C are arranged similar to the three outermost inlets of the FIG. 7B configuration. However, a comparison of these figures illustrates that even a ring of inlets may have a different rotational relationship with the gravity vector (generally used in this disclosure to define vertical). The FIG. 7C configuration corresponds with DPI 500 (see, e.g., FIG. 5B). Those of ordinary skill in the art will appreciate that the exemplary DPIs illustrated in this disclosure may have their inlet and outlet numbers and arrangements varied depending on the intended use. For instance, FIG. 7D shows a DPI much the same as DPI 200 but modified to use the inlet/outlet configuration portrayed by FIG. 7A. As further example, FIG. 7E shows a DPI much the same as DPI 200 but modified to use the inlet/outlet configuration of FIG. 7C.

The lines in FIGS. 7A, 7B, and 7C are non-limiting examples of where the lowest portion of a powder support surface may be positioned relative to the inlet(s) and outlet(s). As the lines illustrate, a powder support surface may be positioned above all inlets and outlets. Alternatively, the powder support surface may be positioned at a height at which part of an inlet and/or outlet is positioned. Alternatively, the powder support surface may be positioned at a height which results in some inlets and/or outlets are higher than the powder support surface but other inlets and/or outlets are lower than the powder support surface. Alternatively, the powder support surface may be positioned at a height such that all inlets and/or outlets are lower than the powder support surface. Recall that the position of the opening through which powder enters the aerosolization chamber relative to the powder support surface plays a significant role in the maximum amount of powder that may enter the aerosolization chamber between actuations.

In some embodiments, a shelf may be adjusted to any of a plurality of different heights (e.g., changed among the height lines indicated in FIGS. 7A-7C) within the aerosolization chamber. In this way a single device may be adjusted based on the particular powder formulation (and respective powder density) as well as total dosage intended for a patient. The shelf height may be manually adjustable. For example, the shelf may be slidable into and out of side wall openings to the aerosolization chamber. When seated in the aerosolization chamber at any one of the available heights, the shelf may physically block any air flow through the corresponding opening. All other openings corresponding to other available heights may be covered, e.g., by a cap of suitable material (e.g., silicone).

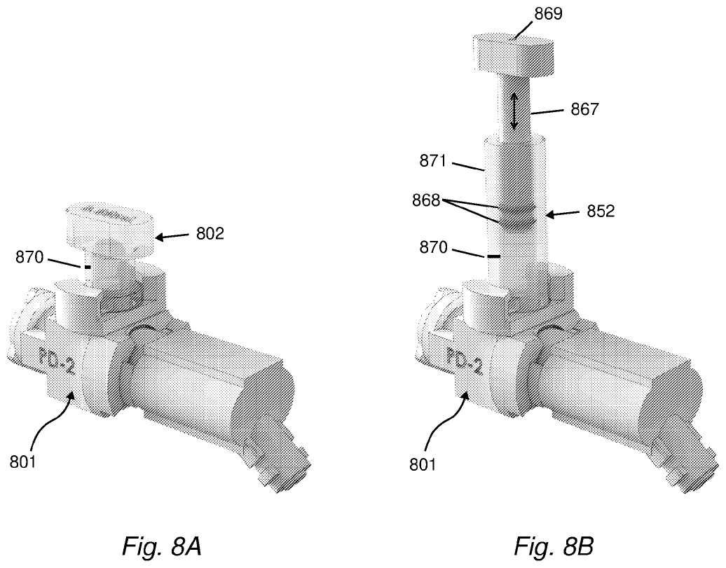

FIGS. 8A and 8B depict non-limiting alternative types of reservoirs 802 and 852 which may be employed with any exemplary DPI. In FIG. 8A, the reservoir 802 has a fixed internal volume which corresponds with a maximum volume of powder which may be loaded into the reservoir 802. By contrast, the reservoir 852 in FIG. 8B has an adjustable total volume.

For consistent performance across a range of loaded doses, an adjustable volume reservoir 852 may be used. An adjustable reservoir such as that of FIG. 8B may be used to eliminate the dead space above the loaded powder. A variety of reservoir volumes may be achieved, for example in the range of 0.5-1.5 mL (even larger upper limits are possible but not generally expected to be needed for many powder inhalation treatments). An adjustable powder reservoir enables a platform to perform consistently with loaded dose between, e.g., 10 mg to 150 mg (based on a bulk powder density of 0.10 g/cm³).

Embodiments with adjustable volume reservoirs may allow for changing the reservoir size in any of a number of different physical configurations. As a non-limiting example, reservoir 852 comprises a sliding insert/plunger 867 which can be moved relative a reservoir body 871 to modify the reservoir volume. Moving the insert 867 reduces, minimizes, or eliminates dead-space above the loaded powder.

Two concentric O-rings 868 at the tip of the insert 867 provide an airtight seal. A hole 869 may be included allowing for venting during position adjustment such that the plunger when sliding to reduce reservoir volume does not force powder out of the reservoir. The hole 869 may be sealed prior to and during actuation, e.g., with a finger or plug. The adjustable insert of the plunger 867 may be positioned just above the resting powder in the reservoir before the first actuation and repositioned after subsequent actuations as needed. The venting hole 869 may be reopened during any positioning of the plunger to allow dead space air to exit through the insert and not compress or force the loaded powder into the aerosolization chamber. An automatic vent sealing device may be used in hole 869 in which opening the vent for positioning may require a push or squeeze button configuration that seals automatically after positioning, or via electromechanical control.

Variable powder doses may be loaded into the powder reservoir in either of FIGS. 8A and 8B. The powder reservoir then connects to the aerosolization chamber 801. This enables the aerosolization chamber 801 to maintain previously established dimensions and flow conditions that have been shown to be conducive for high efficiency aerosolization. The powder reservoirs 802 and 852 may be made of a clear or translucent material to enable the user or administrator to see that the reservoir has been emptied after use, thereby confirming that the dose was delivered. Markings on the powder reservoir may be used to indicate the level of dose and may be useful if the dose is to be filled into the reservoir prior to use.

In reservoirs 802 and 852, as well as reservoirs of any other embodiments of this disclosure, a passage 870 (e.g., a one way passage) may be provided for air to flow into the reservoir for the sole purpose of filling space which develops in the reservoir as powder leaves the reservoir, thereby avoiding any possibly of a partial vacuum developing within the reservoir which could affect powder freely descending out of the reservoir.

### Examples

The following examples demonstrate the effectiveness of various prototypical embodiments consistent with this disclosure. In some of the examples, reference is made to an airway model. For all instances where such model is references, the model refers to a previously developed preterm NT in vitro model. The model is described in:
Howe, C., Momin, M. A., Aladwani, G., Hindle, M., & Longest, P. (2022). Development of a High-Dose Infant Air-Jet Dry Powder Inhaler (DPI) with Passive Cyclic Loading of the Formulation. Pharmaceutical Research, 39, 3317-3330.
and
Howe, C., Momin, M. A., Aladwani, G., Strickler, S., Hindle, M., & Longest, W. (2023). Advancement of a High-Dose Infant Air-Jet Dry Powder Inhaler (DPI) with Passive Cyclic Loading: Performance Tuning for Different Formulations. International Journal of Pharmaceutics, 123199.
   Briefly, the scaled 6-month preterm NT airway model includes flexible nostrils and anterior nose connected to a rigid middle passage, throat and approximately %^{f} of the trachea, which then connects to a custom low-volume filter housing. The setup and assembly includes the NT model and a low-volume filter housing. Briefly, the preterm infant airway geometries were scaled down to an infant with a weight of 1600 g and length (height) of 40.7 cm, based on a high-quality CT scan of 6-month-old infant NT geometry. Using the infant body length (height), an appropriate geometric scaling factor of 0.6 was applied to the 6-month-old NT airway to reduce the model to that of a preterm infant with weight and height of about 1600 g and 40.7 cm, respectively. The resulting preterm airway has a tracheal length and diameter (proximal) of approximately 26 and 3 mm, respectively. While these parameters are known to vary, they do fall within the expected range for preterm infants of 25 to 30 weeks gestational age (GA) based on reported studies.

The scaled 6-month preterm NT model was constructed with twist lock interfaces and O-rings that provided air tight seals and facilitated case of use. The low-volume filter housing accommodated the low AAV of 10 mL used for a preterm infant, with a dead space of only 2.7 mL before the 1.5″ diameter glass-fiber filter. To provide a smooth and accurate internal airway surface, the middle passage and throat sections of the preterm NT model were built using SLA printing with Accura ClearVue resin (3D Systems). The low-volume filter housing was 3D printed using VeroWhitePlus resin. To facilitate nasal interface prong insertion and the formation of an airtight seal, the face and anterior nose section were molded with a skin-like silicone elastomer. The face adapter was printed using VeroWhitePlus resin and was glued to the soft face mold, allowing for a secure and air-tight connection to the rest of the NT model. The three distinct airway regions of the NT model used for regional deposition quantification were anterior nose, middle passage, and throat.

### Example 1. Comparison of Alternative DPI Configurations

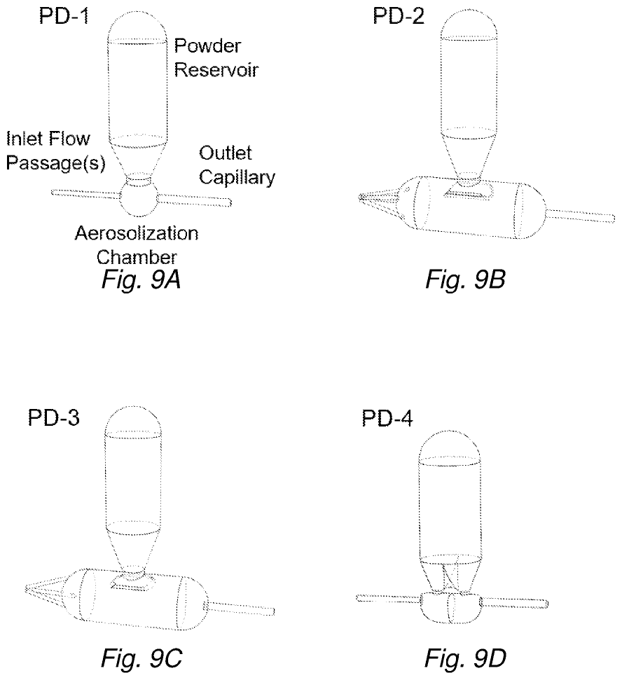

Four alternative air-jet DPIs with a passive cyclic loading design are compared using an initial 10 mg powder mass. These four alternatives are depicted by FIGS. 9A, 9B, 9C, and 9D, respectively. The best designs are treated as those with highest tracheal filter deposition percentage, which estimates the lung delivery efficiency. These designs are then loaded with a 30 mg powder mass and performance is re-examined. The goal of the new designs is to produce similar aerosolization performance and lung delivery efficiencies independent of the loaded powder mass (10 vs 30 mg). A desirable performance threshold is a lung delivery efficiency of 60% or above using a highly dispersible spray-dried model excipient enhanced growth (EEG) formulation and realistic preterm nose-throat model.

A platform consistent with platform 100 was employed. As discussed above, main components of such a platform are the air source, the air-jet DPI, and the interface, specifically a nasal interface in this Example. The air source at baseline conditions delivers an air actuation volume (AAV) of 10 mL bursts at a Q90 flow rate of 1.7 L/min. The prong of the nasal interface was inserted approximately 5 mm into the nose, consistent with short ventilation support nasal prongs and forms an airtight seal with the nostril.

A single nasal interface was used. The nasal interface in this Example had a straight gradually expanding circular cross-section with a length of 48 mm, and a final internal diameter of 3.6 mm. The end of the expansion transitioned to a rigid curved prong, with an inner diameter of 3.6 mm and outer diameter of 4.6 mm. The outer diameter of the prong was based on a Hudson Prong Size 2, commonly used for preterm infant nasal continuous positive airway pressure (CPAP) administration. A gradual exterior taper was included at the base of the prong, forming a wedge to help facilitate an airtight seal with the infant's nostril.

This Example compares four different air-jet DPI designs using a preterm infant nose-throat (NT) in vitro model and a 10 mg loaded powder mass of AS-EEG formulation. The AS-EEG powder formulation included 30:48:20:2% w/w ratio of albuterol sulfate (AS), mannitol, trileucine, and Poloxamer 188, respectively. The platform interfaced with the preterm NT model which led to a custom low-volume filter for approximating lung delivered dose. In this setup, device ED, nasal interface deposition, NT regional depositions, and lung delivery efficiency (represented by aerosol passing through the larynx and a portion of the trachea and depositing on the filter) were assessed, as percentage values of the loaded dose.

All four designs (FIG. 9A-9D) all used a single outlet capillary from the aerosolization chamber (inner diameter of 0.89 mm and a flush or protruding configuration) comprised of a hollow stainless steel (SAE 304) capillary tube. After exiting the aerosolization chamber, this capillary tube included a 37 degree downward bend prior to connection with the nasal interface. All devices used a flush (capillary does not protrude into the aerosolization chamber) outlet configuration with the exception of PD-3, in which the capillary tube protruded into the aerosolization chamber by 0.5 mm. PD-1 and PD-4 used a single inlet of 0.6 mm diameter while PD-2 and PD-3 used multiple inlets with 0.5 mm diameters. These inlet geometries were formed into the structure of the air-jet device during 3D printing. One subtle difference in inlet configurations was that PD-2 had four inlets, all directing the inlet airflow around the initial powder bed. PD-3 had three inlets with one directing a portion of the inlet flow toward the powder bed and the other two directing the airflow through the lower region of the aerosolization chamber.

FIGS. 9A-9D show the four unique air-jet DPIs prototyped to investigate passive cyclic loading for the platform. FIGS. 9A-9D show the internal airway geometries of each air-jet DPI passive design (PD), labeled as PD-1 through PD-4, with basic elements including small diameter inlet flow passage(s), aerosolization chamber, powder reservoir (where the powder was initially loaded), and outlet capillary. In each design, the powder reservoir was positioned above (with respect to gravity) the aerosolization chamber, centered perpendicular to the direction of primary air flow. In this study, the powder reservoir was filled with the desired dose (mass of powder) and then connected to the air-jet DPI with a twist-lock and O-ring seal. The initial standard powder reservoir could accommodate a powder volume up to 0.55 mL. Each air-jet PD had identical connections for powder reservoir attachment, while the inlets, aerosolization chamber geometries, and outlets differed. For the initial comparison of the four unique air-jet DPI designs, the number of actuations to clear the device was also recorded, in which actuations continued until two consecutive visibly clear (no aerosol visible passing through the nasal interface) actuations were observed.

The four designs all used a single outlet capillary from the aerosolization chamber (inner diameter of 0.89 mm and a flush or protruding configuration) comprised of a hollow stainless steel (SAE 304) capillary tube. After exiting the aerosolization chamber, this capillary tube included a 37 degree downward bend prior to connection with the nasal interface. All devices used a flush (capillary does not protrude into the aerosolization chamber) outlet configuration with the exception of PD-3, in which the capillary tube protruded into the aerosolization chamber by 0.5 mm. PD-1 and PD-4 used a single inlet of 0.6 mm diameter while PD-2 and PD-3 used multiple inlets with 0.5 mm diameters. These inlet geometries were formed into the structure of the air-jet device during 3D printing. One subtle difference in inlet configurations was that PD-2 had four inlets, all directing the inlet airflow around the initial powder bed. PD-3 had three inlets with one directing a portion of the inlet flow toward the powder bed and the other two directing the airflow through the lower region of the aerosolization chamber.

Concerning the aerosolization chambers, PD-1 (FIG. 9A) included a small spherical geometry (diameter of 4.8 mm) with a 3 mm diameter connection to the powder reservoir. PD-2 (FIG. 9B) and PD-3 (FIG. 9C) both had a horizontal capsule shaped aerosolization chamber with a volume ~0.68 mL, and a powder shelf or tray placed directly below the powder reservoir to facilitate dose metering. The PD-2 device used a 3 mm diameter opening to the powder reservoir with the shelf positioned to provide an approximate 0.026 mL volume for the powder to rest during each actuation. The PD-3 device used a 2.7 mm diameter opening to the powder reservoir with the shelf positioned to provide an approximate 0.013 mL volume for powder metering. PD-4 (FIG. 9D) used a small aerosolization chamber of ~0.05 mL with two small (1 mm diameter) openings to the powder reservoir, spaced equidistance between the inlet/outlet and center of the chamber (as pictured in FIG. 9D).

Performance of the four unique PD designs was initially evaluated using the 10 mg mass loading of the AS-EEG powder formulation. Table 1 provides the deposition fractions within each region based on the loaded formulation mass, as well as the number of actuations required to provide two consecutive clear actuations (no powder visible exiting the air-jet DPI).

PD-4 with the 1 mm PDTs did not let powder flow under the influence of gravity alone. Instead, powder was pulled into the aerosolization chamber and air stream therein only during actuation. While PD-4 therefore exhibited a type of passive loading, it did not exhibit gravity fed passive loading. Performance of PD-4 was not as good as PD-2 and PD-3, both of which exhibited gravity fed passive loading.

PD-2 and PD-3 produced the lowest DPI retention (~11-13%) and the highest tracheal filter deposition of ~60%, which met the goal of achieving 60% estimated lung delivery efficiency. PD-2 and PD-3 also had statistically similar performance across all deposition regions with the only difference being that PD-2 required one extra actuation to clear the device. PD-4 did not perform as well as the other devices and had the highest variability, based on mean (SD) DPI retention and tracheal filter delivery of 33.2 (5.5) % and 46.2 (2.1) %, respectively. This device also required the highest number of actuations. PD-1 had a mid-range performance with a mean tracheal filter deposition of ~53% and a DPI retention ~17%. PD-1 was also in the mid-range in terms of consistency with larger standard deviations than PD-2 and PD-3, but less than seen with PD-4. Due to the similar and best performance of PD-2 and PD-3, both devices were selected for sensitivity analysis of loaded dose in the next step.

TABLE 1
Lung delivery efficiencies (estimated as Tracheal Filter %) and regional
deposition fractions (based on 10 mg loaded dose) for the AS-EEG
formulation and an initial round of different device designs.

| | PD-1 | | PD-2 | | PD-3 | | PD-4 | |
|---|---|---|---|---|---|---|---|---|
| | # of Actuations | | | | | | | |
| Deposition Region | 4 | | 5 | | 4 | | 6 | |
| DPI Retention (%)^{a} | 16.8 | (4.8) | 12.9 | (1.1) | 10.9 | (0.7) | 33.2 | (5.5)^{b} |
| Nasal Interface (%) | 5.2 | (0.4) | 4.3 | (0.2) | 4.6 | (0.3) | 4.0 | (1.1) |
| Total ED (%)^{a} | 77.9 | (4.6) | 82.8 | (1.2) | 84.5 | (0.4) | 62.8 | (5.5)^{b} |
| Anterior Nose (%) | 4.8 | (1.0) | 5.4 | (1.6) | 4.5 | (0.3) | 3.2 | (1.1) |
| Middle Passage (%)^{a} | 7.8 | (0.9) | 7.8 | (0.4) | 8.4 | (1.4) | 5.0 | (1.3)^{b} |
| Throat (%) | 11.5 | (3.1) | 9.1 | (0.9) | 10.4 | (1.3) | 6.3 | (4.6) |
| Total NT (%) | 24.1 | (4.9) | 22.3 | (1.1) | 23.2 | (2.1) | 14.4 | (7.0) |
| Tracheal Filter (%)^{a} | 53.1 | (1.7) | 60.0 | (0.6)^{b} | 60.9 | (1.9)b | 46.2 | (2.1)^{b} |

Mean values with standard deviations (SD) shown in parenthesis, n = 3.
^{a}p < 0.05 significant effect of design on deposition region (one-way ANOVA).
^{b}p < 0.05 significant difference compared to PD-1 case (post-hoc Tukey).
DPI, dry powder inhaler; ED, emitted dose; NT, nose-throat; PD, passive design.

Two of the designs (PD-2 and PD-3) resulted in improved estimated lung delivery efficiencies over the previously developed system (e.g., Howe C, Momin MA, Farkas DR, Bonasera S, Hindle M, and Longest P. Advancement of the Infant Air-Jet Dry Powder Inhaler (DPI): Evaluation of Different Positive-Pressure Air Sources and Flow Rates. Pharmaceutical Research. 2021; 38:1615-1632 with ~50% lung delivery efficiency), while also enabling high dose delivery. Both PD-2 and PD-3 performed similarly when loaded with a 10 mg mass of AS-EEG formulation, and were able to deliver ~60% of the loaded dose to the tracheal filter through a preterm NT model. Based on the observation that tripling the loaded dose did not have an impact on lung delivery efficiency, it is to be expected that mass loadings can be increased even further without negative impacts. Furthermore, tripling the volume of the powder reservoir resulted in only a minor reduction in lung delivery efficiency from ~60% to ~55%. As a result, the passive cyclic loading strategy appears capable of delivering a range of drug masses from ~10 mg through ~150 mg (at a powder bulk density of 0.1 g/cm³) with only minor changes in lung delivery efficiency. Considered collectively, results of this Example indicate that the air-jet DPI with passive cyclic loading is expected to exhibit consistent performance across a range of loaded doses and is largely insensitive to pulmonary mechanics.

In conclusion, a passive cyclic loading approach was successfully implemented for the infant air-jet DPI platform. During in vitro testing using a preterm NT model and AS-EEG formulation, the platform performed consistently when the loaded dose increased from 10 mg to 30 mg and also when connected to the pulmonary mechanics outlet condition. The optimal PD devices (PD-2 and PD-3) achieved an estimated lung delivery efficiency of ~60% when administered through a 1600 g preterm NT model.

### Example 2. Impact of Loaded Dose

The above-described test of Example 1 was reperformed for PD-2 and PD-3 at 30 mg powder mass loading instead of 10 mg to determine potential sensitivity to powder loading. All experimental procedures remained the same as described above except for the loading of 30 mg of powder instead of 10 mg. Likewise, actuations continued until no powder was visible passing through the system and one subsequent clear actuation was performed. The total number of actuations used in all cases was recorded. For PD-2 and PD-3, Table 2 compares the results with 10 mg powder mass loadings from Table 1 with additional data using 30 mg loadings. For both designs, there was a slight but statistically significant reduction is nasal interface deposition (~2% absolute difference) with the larger loaded dose. PD-3 also produced a slight but statistically significant reduction in the anterior nose deposition region (~1%) with the larger loaded dose. All other regions for both designs remained statistically equivalent, indicating these two designs are not sensitive to the loaded dose for the values tested. It is also observed that the dose delivered on each actuation appears to be consistent. Due to the experimental procedure of delivery, requiring two consecutive visibly clear actuations before ending the trial, it can be inferred that performance will remain similar when PD-2 is actuated 3 and 9 times, and while PD-3 is actuated 2 and 6 times, for a 10 and 30 mg powder mass loading, respectively. For both designs, a 3-fold increase in powder mass also required a 3-fold increase in number of actuations to empty the device, which indicates a similar dose per actuation between the two loaded dose masses.

TABLE 2
Lung delivery efficiencies (estimated as Tracheal Filter
%) and regional deposition fractions (based on loaded
dose) for the AS-EEG formulation and comparisons of
10 vs 30 mg loaded powder in lead devices.

| | PD-2 | | | | PD-3 | | | |
|---|---|---|---|---|---|---|---|---|
| | 10 mg | | 30 mg | | 10 mg | | 30 mg | |
| Deposition | # of Actuations | | | | | | | |
| Region | 5 | | 11 | | 4 | | 8 | |
| DPI | 12.9 | (1.1) | 16.1 | (2.1) | 10.9 | (0.7) | 12.2 | (2.9) |
| Retention (%) | | | | | | | | |
| Nasal | 4.3 | (0.2) | 2.4 | (0.1)^{a} | 4.6 | (0.3) | 2.8 | (0.4)^{a} |
| Interface (%) | | | | | | | | |
| Total ED (%) | 82.8 | (1.2) | 81.5 | (2.0) | 84.5 | (0.4) | 85.0 | (3.3) |
| Anterior | 5.4 | (1.6) | 3.3 | (0.3) | 4.5 | (0.3) | 3.7 | (0.5)^{a} |
| Nose (%) | | | | | | | | |
| Middle | 7.8 | (0.4) | 8.2 | (0.3) | 8.4 | (1.4) | 8.3 | (0.5) |
| Passage (%) | | | | | | | | |
| Throat (%) | 9.1 | (0.9) | 10.6 | (0.9) | 10.4 | (1.3) | 12.3 | (1.3) |
| Total NT (%) | 22.3 | (1.1) | 22.1 | (1.0) | 23.2 | (2.1) | 24.4 | (1.3) |
| Tracheal | 60.0 | (0.6) | 58.5 | (1.3) | 60.9 | (1.9) | 60.5 | (1.6) |
| Filter (%) | | | | | | | | |

Mean values with standard deviations (SD) shown in parenthesis, n = 3.
^{a}p < 0.05 significant difference in deposition region compared with 10 mg case (t-test).

### Example 3. Impact of Downstream Pulmonary Mechanics

To test sensitivity of the platform to downstream pulmonary mechanics, the PD-3 air-jet DPI design was chosen and retested while connected to an infant Michigan Lung simulator (Adult/Infant Model, Michigan Instruments, Grand Rapids, MI). Two NT model outlet conditions were considered: one as the standard filter only (used in all other experiments) and then also including the downstream pulmonary mechanics, connected to the lung simulator. This experiment was performed to directly compare the large dose loading condition including downstream pulmonary mechanics in a breathing lung simulator. Using the air source operated at 1.7 L/min, and a 30 mg powder mass loading, the platform was actuated during the inhalation cycle of the lung simulator, while all other methods remained the same. Pulmonary mechanics (PM) considered in this study were lung compliance (mL/cm H₂O), airway resistance (cm H₂O/L/s), breath cycle (sec), and tidal volume (mL). The compliance, breath cycle, and tidal volume were manually set on the lung simulator, and a custom resistance orifice adapter was used to adjust the airway resistance. Air flow from exhalation of the lung simulator was prevented from passing back through the filter and detaching any deposited powder. The use of one-way valves and a bifurcation downstream of the resistance orifice allowed for venting the exhalation gas.

The infant lung simulator was set to its lowest compliance setting of 1 mL/cm H₂O. The inspiratory time was set to 0.5 sec, with a 1 sec breath hold and exhalation period resulting in a ~2.5 sec breath cycle. With the direct-to-infant delivery protocol used in this study, one nostril was connected to the device while the contralateral nostril and mouth were held closed enabling the use of a breath hold period. The tidal volume was adjusted to approximately 10-11 mL. For infants with RDS, airway resistance values have been reported between 100-200 cm H₂O/L/s. An adjustable resistance orifice was built to set the desired airway resistance downstream of the filter. To measure airway resistance through the filter housing and orifice, a pressure sensor (SSCDLNN040MBGSA5, Honeywell, Sensing and Control, Golden Valley, MN) was placed anterior to the filter housing while the neonatal flow meter (Sensirion SFM3400) was placed further upstream. The downstream side of the resistance orifice was opened to atmospheric pressure while a steady upstream flow rate of 2 L/min was set. The pressure was recorded while the orifice was adjusted to provide a total airway resistance expected for an infant with RDS. The resulting calculated resistance was 172 cm H₂O/L/s which falls within the range of expected values.

The analysis of this Example utilized the results of PD-3 with a 30 mg powder mass loading of the AS-EEG formulation (from Table 2; denoted filter-only), compared to a pulmonary mechanics (PM) outlet condition. All other parameters of the experiment were identical, including number of actuations. The comparison of regional drug deposition for filter-only and PM outlet conditions is presented in Table 3. Performance across all deposition regions was statistically similar except for the nasal interface, which decreased by ~-1% (absolute difference). The PM outlet condition was not found to have a statistically significant effect on the estimated lung deliver efficiency, which remained about 60%, but demonstrated an increasing trend in lung delivery efficiency (i.e., 60.5% filter only vs. 62.6% PM outlet). Overall, the performance was found to be insensitive to the addition of the PM outlet condition.

TABLE 3
Lung delivery efficiencies (estimated as Tracheal Filter %) and
regional deposition fractions (based on loaded dose) of the AS-
EEG formulation and 30 mg dose loadings with the PD-3 device and
filter-only vs. pulmonary mechanics (PM) outlet conditions.

| | | |
|---|---|---|
| Deposition Region | Filter-Only | PM |

| | | | | |
|---|---|---|---|---|
| DPI Retention (%) | 12.2 | (2.9) | 10.5 | (1.1) |
| Nasal Interface (%) | 2.8 | (0.4) | 1.7 | (0.5)^{a} |
| Total ED (%) | 85.0 | (3.3) | 87.8 | (1.4) |
| Anterior Nose (%) | 3.7 | (0.5) | 3.7 | (0.4) |
| Middle Passage (%) | 8.3 | (0.5) | 7.9 | (0.9) |
| Throat (%) | 12.3 | (1.3) | 12.3 | (0.7) |
| Total NT (%) | 24.4 | (1.3) | 23.9 | (0.7) |
| Tracheal Filter (%) | 60.5 | (1.6) | 62.6 | (1.4) |

Mean values with standard deviations (SD) shown in parenthesis, n = 3.
^{a}p < 0.05 significant difference between filter-only and PM outlet condition for nasal interface deposition (t-test).
PM, pulmonary mechanics

### Example 4. Impact of Powder Reservoir Volume

Powder reservoir volume sensitivity was analyzed using the PD-3 device and 10 mg powder mass loading with the AS-EEG formulation. A comparison of the initial results using the standard powder reservoir (Table 2 data) with additional data using an extended volume powder reservoir is shown in Table 4. Both powder reservoirs had similar geometry with a half capsule shape of 7.1 mm diameter, differing by length only. The standard powder reservoir had a loading volume of 0.55 mL, while the extended volume was 1.5 mL, and both reservoirs were fabricated using the SLA method to produce clear parts for viewing of powder behavior during actuation. All other parameters remained the same as the initial experimental set. The standard powder reservoir could accommodate dose loadings between 10-50 mg of the AS-EEG formulation used in this study, which had a bulk density of ~0.1 g/cm³ and up to ~150 mg with the extended version.

The extended volume was not found to have an impact on the deposition found in the nasal interface and nose-throat regions; however, a statistically significant difference was found for the DPI retention (also affecting Total ED) and the tracheal filter. The mean DPI retention increased from 10.9% to 15.5% while the mean tracheal filter deposition decreased from 60.9% to 54.5% with the extended powder reservoir. The nearly 3-fold increase of dead space in the powder reservoir was found to impact the performance of the system by lowering the emitted dose and consequently the tracheal filter deposition by approximately 5% (absolute difference).

Assuming that 1.5 mg/kg of PL will be effective in a human infant, the required Surfactant-EEG powder mass for a body weight of 1.6 kg would be ~20 mg, factoring in 25% PL loading in the formulation and a 50% lung delivery efficiency. Assuming the same conditions, the powder mass loading for a full-term infant with a weight of 3.55 kg would be ~44 mg. Based on the results of this Example, the infant air-jet DPI with passive cyclic loading appears capable of delivering this range of powder dose with minimal expected change in lung delivery performance. Through the use of an expanded powder reservoir, the passive cyclic loading approach may also be used to deliver powder versions of these much higher doses with a single loading of the device and at a controlled rate.

TABLE 4
Lung delivery efficiencies (estimated as Tracheal Filter %) and
regional deposition fractions (based on loaded dosc) for the
AS-EEG formulation and comparisons of standard (0.55 mL) vs extended
(1.5 mL) powder reservoir volume with the PD-3 device.

| | | |
|---|---|---|
| Deposition Region | Standard | Extended |

| | | | | |
|---|---|---|---|---|
| DPI Retention (%) | 10.9 | (0.7) | 15.5 | (0.9)^{a} |
| Nasal Interface (%) | 4.6 | (0.3) | 4.5 | (0.3) |
| Total ED (%) | 84.5 | (0.4) | 79.9 | (1.1)^{a} |
| Anterior Nose (%) | 4.5 | (0.3) | 4.9 | (0.8) |
| Middle Passage (%) | 8.4 | (1.4) | 9.6 | (0.2) |
| Throat (%) | 10.4 | (1.3) | 11.1 | (0.8) |
| Total NT (%) | 23.2 | (2.1) | 25.6 | (1.0) |
| Tracheal Filter (%) | 60.9 | (1.9) | 54.5 | (2.3)^{a} |

Mean values with standard deviations (SD) shown in parenthesis, n = 3.
^{a}p < 0.05 significant difference between standard and extended chamber volume (t-test).

### Example 5. Platform Performance with AS-EEG Formulation. Initial platform performance was determined using a preterm infant NT in vitro model and a 10 mg loaded powder mass of AS-EEG formulation. The platform interfaces with the preterm NT model, which leads to a custom low-volume filter for approximating lung delivered dose. The nasal interface used matches the description given above in Example 1. In this setup, device ED, nasal interface deposition, NT regional depositions, and lung delivery efficiency (represented by aerosol passing through the larynx and upper portion of the trachea and depositing on the filter) were assessed as percentage values of the loaded dose. Aerosol characteristics of the air-jet DPI (excluding nasal interface), in terms of mass median aerodynamic diameter (MMAD) and fine particle fractions (FPFs), were determined through cascade impactor testing using a Next Generation Impactor (NGI). The main portion of this study then explored dose metering element configurations across multiple EEG formulations to achieve best or similar performance compared to the model AS-EEG formulation. Preliminary testing revealed that both powder shelf volume, which controls the amount of powder available for aerosolization with each actuation, and powder delivery tube (PDT) diameter, which also controls loading of the powder shelf, were significant design attributes

In Examples 1 and 2 above, the PD2 platform achieved 60% lung delivery efficiencies with both 10 and 30 mg powder loading of the AS-EEG formulation when operated at 1.7 L/min. Previous investigations of surfactant based EEG formulation indicated a higher flow rate produced improved aerosolization (around 4 L/min), so for a baseline comparison in this Example, the AS-EEG formulation was characterized at a flow rate of 4 L/min. Triplicate runs were performed for both the preterm infant NT model and NGI sizing of aerosol. In all cases, the PD2 air-jet DPI with 10 mg of powder loaded in the standard powder reservoir was actuated five times with a flow rate of 4 L/min. Performance and characteristics of the aerosol are presented in Table 5, which provides the deposition fractions within each region based on the loaded formulation mass and the sizing properties of the aerosol as emitted from the air-jet DPI outlet. When operated at the 4 L/min flow rate, the PD2 platform produced a tracheal filter deposition of ~45% (based on loaded dose) and a mean MMAD of 1.63 μm, while the FPFs for <5 and <1 μm were ~85% and ~25% (based on NGI recovered dose), respectively.

TABLE 5
Lung delivery efficiencies (estimated as Tracheal Filter %)
and regional deposition fractions (based on 10 mg loaded
dose) for the AS-EEG formulation and NGI determined
particle size characteristics from the PD2 platform.
PD2

| Deposition Region | | | Size Characteristics | | |
|---|---|---|---|---|---|
| DPI Retention (%) | 15.8 | (3.0) | DPI Retention (%) | 16.5 | (1.5) |
| Nasal Interface (%) | 5.8 | (1.0) | | | |
| Total ED (%) | 78.4 | (2.4) | MMAD (μm) | 1.63 | (0.07) |
| Anterior Nose (%) | 7.2 | (0.3) | GSD | 2.53 | (0.28) |
| Middle Passage (%) | 10.2 | (0.4) | FPF_{5 μm} (%) | 84.7 | (2.1) |
| Throat (%) | 14.0 | (1.7) | FPF_{1 μm} (%) | 24.9 | (2.1) |
| Total NT (%) | 31.4 | (1.2) | | | |
| Tracheal Filter (%) | 45.2 | (2.2) | | | |

Mean values with standard deviations (SD) shown in parenthesis, n = 3.
All experiments used 5 actuations.
DPI, dry powder inhaler; ED, emitted dose; FPF, fine particle fraction; GSD, geometric standard deviation; MMAD, mass median aerodynamic diameter; NT, nose-throat; PD, passive design.

### Example 6. Adjustable Powder Reservoir

To enable consistent performance across a range of loaded doses, an adjustable volume reservoir design was investigated with a maximum loaded volume of 1.5 mL. In this design, a sliding insert plunger was used to modify the reservoir volume and minimize dead-space above the loaded powder. FIG. 8A shows the standard volume reservoir (0.55 mL). FIG. 8B shows the adjustable volume reservoir (up to 1.5 mL). For a smooth interior wall, the adjustable powder reservoir was constructed in the same manner as the standard volume reservoir, using SLA printing with Accura ClearVue resin (3D Systems). The initial prototype of the adjustable insert was 3D printed using VeroWhitePlus resin with two concentric O-rings at the tip to provide an airtight seal and a central hole allowing for venting during position adjustment. This central hole was sealed manually by the operator's index finger during device actuation. The adjustable insert was positioned just above the resting powder before the first actuation and allowed for repositioning after subsequent actuations as needed. The venting hole remained open during positioning to allow dead space air to exit through the insert and not compress or force the loaded powder into the aerosolization chamber. While the procedures for the initial test design were manageable in a laboratory setting, a commercial embodiment may utilize an automatic vent sealing scheme in which opening the vent for positioning is achievable with a push or squeeze button configuration that seals automatically after positioning.

Table 6 compares the baseline (standard) results from Table 5 with additional data using the adjustable powder reservoir and 10 mg powder mass loadings. Drug recovery in all regions between the two configurations remained statistically equivalent, indicating the adjustable powder reservoir approach enabled a larger fillable volume without degrading performance. As the platform has been shown to be insensitive to loaded dose (10 to 30 mg) (Examples 1 and 2 above) and now insensitive to the adjustable powder reservoir design (Table 6), continuation with 10 mg powder loadings and the initial fixed-volume powder reservoir (0.55 mL) were deemed reasonable.

TABLE 6
Lung delivery efficiencies (estimated as Tracheal Filter %) and
regional deposition fractions (based on loaded dose) for the
AS-EEG formulation and comparisons of 0.55 mL (standard) vs
1.5 mL (adjustable) powder reservoir with the PD2 platform.

| | | |
|---|---|---|
| Deposition Region | Standard | Adjustable |

| | | | | |
|---|---|---|---|---|
| DPI Retention (%) | 15.8 | (3.0) | 15.6 | (2.8) |
| Nasal Interface (%) | 5.8 | (1.0) | 3.7 | (0.4) |
| Total ED (%) | 78.4 | (2.4) | 80.7 | (2.8) |
| Anterior Nose (%) | 7.2 | (0.3) | 6.3 | (1.4) |
| Middle Passage (%) | 10.2 | (0.4) | 11.0 | (2.0) |
| Throat (%) | 14.0 | (1.7) | 15.7 | (2.8) |
| Total NT (%) | 31.4 | (1.2) | 33.0 | (5.8) |
| Tracheal Filter (%) | 45.2 | (2.2) | 45.5 | (4.0) |

Mean values with standard deviations (SD) shown in parenthesis, n = 3.
All experiments used 5 actuations.
No significant difference found between deposition regions (t-test)

### Example 7. Device Emptying Characterization

To address concerns of delivering a large dose too quickly or inconsistently over the course of multiple actuations, a device emptying characterization study was performed with the AS-EEG formulation. An aerosol collection assembly was designed and 3D printed using VeroWhitePlus resin and was assembled with intermediate O-ring seals. The air-jet DPI was inserted into the collection assembly which allowed the aerosol exiting the outlet of the air-jet DPI to be collected on the glass-fiber filter as co-flow room air was pulled through the collection assembly to provide a uniform flow of 45 L/min. The emptying characterization was performed in triplicate for both 10 mg and 30 mg powder masses. For the 10 mg loaded dose, the air-jet DPI was actuated five times, while the 30 mg loaded dose required 10 actuations. Between each actuation of the air-jet DPI, the collection assembly was opened to replace the filter so that the emitted dose for each actuation could be quantified individually.

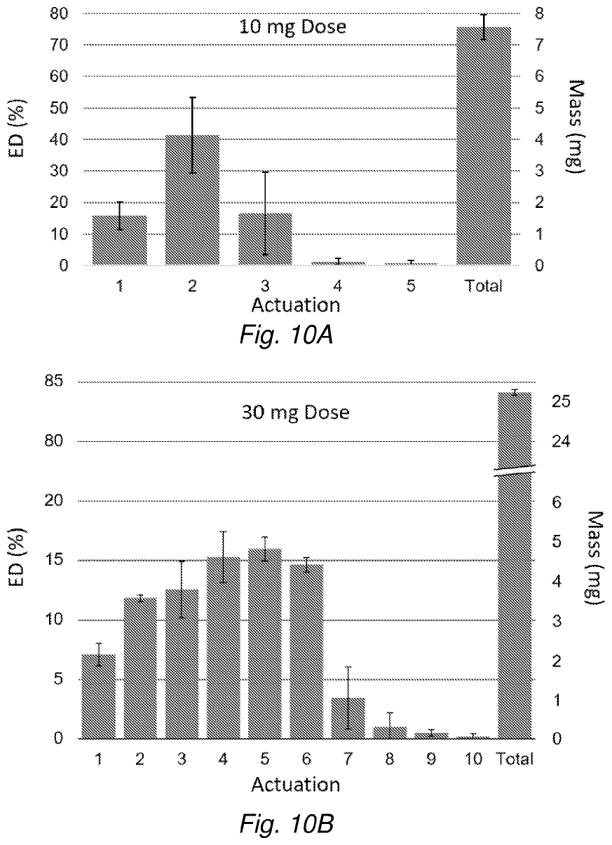

A design goal for the infant air-jet DPI platform is to limit the maximum amount of powder dose that may be delivered per actuation. An initial experiment utilized the device ED setup described at the outset of this Example to determine the emitted dose per actuation for a 10 mg powder mass loading. For the 10 mg loading, the device was actuated five times into five separate collection filters, with the results plotted in FIG. 10A. As expected, nearly all of the total ED was emitted in the first 3 actuations; however, dose/actuation was not equivalent. The mean ED for the first and third actuations were nearly the same at about 15% or 1.5 mg, while the mean ED for the second actuation was over twice as large at about 40% or 4.0 mg. The trend in ED per actuation indicted a startup phase, peak at ~4 mg/actuation, and then a rapid reduction.

To determine if the ED per actuation plateaus with higher dose loadings, a second experiment using a 30 mg powder mass was performed. With an identical setup, 30 mg of powder was loaded and 10 actuations were performed into 10 separate collection filters. The results of the 30 mg loading are plotted in FIG. 10B, in which the ED per actuation does appear to plateau around 4.5-5 mg. The trends for both cases indicate the ED per actuation has a startup process in the initial actuations followed by a plateau and then a rapid drop off. While there is some variability, it is expected to hold that device emptying will occur with 3 actuations per 10 mg of loaded powder, and the maximum ED per actuation is about 5 mg over the course of a treatment.

Evaluating the device emptying characteristics also led to positive results, in which a maximum ED per actuation of ~5 mg was observed. While the ED per actuation was not identical across the entire treatment (a startup period was observed), the overall total ED had small deviations and a plateau effect was seen. For example, with 30 mg of loaded powder, the mean total ED for three trials was 84.1% with a SD of 0.88%, and the three highest Eds per actuation were 4.4, 4.6 and 4.8 mg. A <5 mg per actuation dose is expected to be safe based on the low NT and tracheal deposition values and disperse deposition patterns observed in this Example.

### Example 8. Metering Element Tuning for Different Powder Formulations

Passive metering system elements were investigated and tuned to produce similar performance (in terms of estimated lung delivery efficiencies) across three different powder formulations. Finally, the performance and aerosolization characteristics of best case configurations for each formulation were determined and compared.

The baseline PD2 device considered in Tables 5 and 6 had a shelf position that allowed ~26 μL (i.e., 0.026 mL) of formulation to reside in the space between the PDT (diameter=3 mm) outlet and shelf. This baseline device is referred to as PD2-26. To test the impact of shelf volume with the AS-EEG formulation at an actuation flow rate of 4 L/min, larger and smaller shelf volumes of ~36 μL (PD2-36) and ~8 μL (PD2-08) were tested. Surprisingly, there was nearly no effect on performance with the different shelf volumes and no statistically significant effect on tracheal filter deposition. As such, the original PD2-26 was used for initial baseline evaluations with other formulations.

The formulation designated AS-EEG was already described above. Survanta® based Surfactant-EEG powders were prepared by spray drying of the feed dispersions containing Survanta®, mannitol, sodium chloride and 1-leucine at a ratio of 40:30:10:20% w/w. For further comparison, synthetic lung surfactant powder formulation (DPPC-BYL3-EEG) was prepared by spray drying of feed dispersions containing DPPC, B-YL, hygroscopic excipients (mannitol and sodium chloride) and dispersion enhancer (1-leucine) (at a ratio of 25:3:42:10:20).

Geometric sizes of primary particles can be approximated based on the laser diffraction data at a 4 bar dispersion pressure. Mean Dv50 values are reported for each formulation in Table 7. Primary particle sizes appear similar between the AS and Survanta-EEG formulations and are smaller for the DPPC-BYL3-EEG formulation. Bulk density values indicate large differences among the powders and can be ranked from most dense to least dense as Survanta-EEG>AS-EEG>DPPC-BYL3-EEG (Table 7). These bulk density values will impact the mass of powder that can be held within the fixed volume of each powder shelf.

TABLE 7
Powder properties for each formulation based on primary
particle size (geometric) and approximate bulk density.

| | | Survanta- | DPPC- |
|---|---|---|---|
| | AS-EEG | EEG | BYL3-EEG |
| DV50 (μm) | 1.13 (0.03) | 1.15 (0.03) | 1.03 (0.00) |
| Bulk Density (g/cm³) | 0.10 | 0.18 | 0.04 |

Initial results of the PD2-26 device and Survanta-EEG formulation produced a low lung delivery efficiency ~20%, as reported in Table 8. One of the driving factors for the lower performance was likely the higher density of the formulation and associated higher mass aerosolized with each actuation. The PD2-26 shelf volume allowed ~2.6 mg of the AS-EEG powder (~0.10 g/cm³) to fall and rest on the shelf for each actuation and ~4.7 mg of the Survanta-EEG powder (~0.18 g/cm³), nearly double that of the AS-EEG powder. By reducing the shelf gap distance (between the PDT outline and shelf) and likewise the amount of powder able to rest on the shelf between actuations, performance may be improved. Various decreasing shelf volumes were investigated (~20 (not shown), ~16, and ~8 μL) and the best performance was observed with a shelf volume ~16 μL (PD2-16), which equates to ~2.9 mg of Survanta-EEG powder able to rest on the shelf between actuations (Table 8). Reducing the shelf volume for the Survanta-EEG formulation nearly doubled the estimated lung delivery efficiency to ~38% compared with the baseline case (~20%).

A secondary set of screenings was then performed in which the diameter of the PDT was decreased to further limit the amount of powder per actuation. A final configuration with a shelf volume ~12 μL and an opening diameter of 1.5 mm (PD2-12-1.5) was found to produce a desired tracheal filter deposition of ~47%, which closely matched the performance of the AS-EEG formulation (Table 8).

TABLE 8
Lung delivery efficiencies (estimated as Tracheal Filter
%) and regional deposition fractions (based on loaded
dose) for the Survanta-EEG formulation and comparison
of different passive metering element configurations.

| Deposition | | | | | | | PD2- | |
|---|---|---|---|---|---|---|---|---|
| Region | PD2-26 | | PD2-16 | | PD2-08 | | 12-1.5 | |
| DPI | 15.4 | (1.8) | 7.7 | (0.5)^{b} | 9.9 | (1.2)^{b} | 10.4 | (1.7)^{b} |
| Retention (%)^{a} | | | | | | | | |
| Nasal | 3.2 | (0.3) | 4.9 | (1.4) | 3.4 | (0.4) | 4.7 | (0.7) |
| Interface (%) | | | | | | | | |
| Total ED (%)^{a} | 81.4 | (2.1) | 87.4 | (1.7)^{b} | 86.7 | (1.0)^{b} | 84.8 | (1.0) |
| Anterior | 13.3 | (1.7) | 12.5 | (1.7) | 12.6 | (1.2) | 9.2 | (1.2)^{b} |
| Nose (%)^{a} | | | | | | | | |
| Middle | 19.1 | (2.2) | 13.5 | (4.2) | 15.3 | (4.5) | 15.2 | (2.6) |
| Passage (%) | | | | | | | | |
| Throat (%)^{a} | 18.9 | (1.8) | 18.2 | (0.8) | 21.0 | (2.6) | 13.9 | (1.4)^{b} |
| Total NT (%)^{a} | 51.3 | (2.0) | 44.2 | (6.1) | 48.9 | (5.7) | 38.3 | (2.4)^{b} |
| Tracheal | 23.7 | (0.7) | 38.0 | (4.0)^{b} | 35.4 | (2.4)^{b} | 47.0 | (0.2)^{b} |
| Filter (%)^{a} | | | | | | | | |

Mean values with standard deviations (SD) shown in parenthesis, n = 3.
All experiments used 5 actuations.
^{a}p < 0.05 significant effect of design on deposition region (one-way ANOVA).
^{b}p < 0.05 significant difference compared to PD2-26 (post-hoc Tukey).

Initial results of the PD2-26 device and DPPC-BYL3-EEG formulation produced a high lung delivery efficiency of ~43%, but when increasing the shelf volume (PD2-40) to account for the lower density (~0.04 g/cm³), performance was not improved (data not shown). Also, emptying the device required 9-10 actuations as opposed to only 3-5 actuations with the AS-EEG formulation. For a small dose (~10-20 mg), this is likely acceptable, but for large doses (~90 mg), an extended delivery time may not be desirable. For example, an actuation (~0.2 s), breath hold period (~5 s) and exhalation (~<1 s) produce an approximate 6 s cycle per actuation. With the AS-EEG formulation requiring 3 actuations per 10 mg, a 90 mg loaded powder dose would take less than 3 min to administer; whereas, the DPPC-BYL3-EEG formulation requiring 10 actuations per 10 mg would take about 9 min to administer a 90 mg dose. To increase the speed of delivery, the diameter of the PDT was increased instead of adjusting the shelf volume. The initial diameter of 3 mm (with the PD2-26 device) was increased to 3.5 mm, and the device labeled as PD2-26-3.5. The resulting configuration was able to deliver the 10 mg loaded dose in ~3-5 actuations while providing a high lung delivery efficiency of ~47%, matching the baseline performance of the AS-EEG formulation. Using the 3.5 mm diameter opening, a larger shelf volume was again tested for the lower density powder (PD2-40-3.5 design); however, improvements were not seen. Table 9 presents the tested configurations for the DPPC-BYL3-EEG formulation, in which modification to the reservoir opening diameter alone was enough to tune the device to match the baseline AS-EEG performance.

TABLE 9
Lung delivery efficiencies (estimated as Tracheal Filter
%) and regional deposition fractions (based on loaded
dose) for the DPPC-BYL3-EEG formulation and comparison
of different passive metering element configurations.

| | PD2-26 | | PD2-26-3.5 | | PD2-40-3.5 | |
|---|---|---|---|---|---|---|
| | # of Actuations | | | | | |
| Deposition Region | 10 | | 5 | | 5 | |
| DPI Retention (%) | 12.2 | (2.4) | 12.2 | (2.0) | 12.1 | (1.7) |
| Nasal Interface (%) | 3.6 | (0.5) | 4.0 | (0.6) | 4.4 | (1.2) |
| Total ED (%) | 84.2 | (2.9) | 83.8 | (2.3) | 83.5 | (0.6) |
| Anterior Nose (%) | 8.9 | (1.3) | 7.3 | (0.4) | 10.1 | (1.8) |
| Middle Passage (%) | 11.6 | (1.6) | 11.9 | (2.0) | 13.1 | (1.4) |
| Throat (%) | 15.5 | (3.5) | 14.1 | (2.3) | 14.9 | (2.3) |
| Total NT (%) | 35.9 | (6.4) | 33.3 | (4.3) | 38.0 | (1.9) |
| Tracheal Filter (%) | 43.0 | (9.2) | 46.7 | (1.4) | 41.1 | (1.9) |

Mean values with standard deviations (SD) shown in parenthesis, n = 3.
No significant effect of design on deposition region (one-way ANOVA).

The best device configurations for each formulation based on the testing performed in this study are considered to be PD2-26, PD2-12-1.5 and PD2-26-3.5 for the AS-EEG, Survanta-EEG and DPPC-BYL3-EEG formulations, respectively. Table 10 presents the regional deposition fractions from the in vitro preterm NT model testing for each formulation using the relative best configuration air-jet DPI. With the two passive metering elements (shelf height/volume and PDT diameter), it was possible to tune both surfactant formulations to match (no statistically significant differences) the base-line AS-EEG formulation performance for most performance metrics and most importantly for the estimated lung delivery efficiency (~45-47% of loaded dose).

TABLE 10
Lung delivery efficiencies (estimated as Tracheal Filter
%) and regional deposition fractions (based on loaded
dose) for each formulation with 10 mg powder loadings
and relative best metering element configuration.

| | | | Survanta- | | DPPC- | |
|---|---|---|---|---|---|---|
| Deposition Region | AS-EEG | | EEG | | BYL3-EEG | |
| DPI Retention (%) | 15.8 | (3.0) | 10.4 | (1.7) | 12.2 | (2.0) |
| Nasal Interface (%) | 5.8 | (1.0) | 4.7 | (0.7) | 4.0 | (0.6) |
| Total ED (%)^{a} | 78.4 | (2.4) | 84.8 | (1.0)^{b} | 83.8 | (2.3)^{b} |
| Anterior Nose (%)^{a} | 7.2 | (0.3) | 9.2 | (1.2)^{b} | 7.3 | (0.4) |
| Middle Passage (%)^{a} | 10.2 | (0.4) | 15.2 | (2.6)^{b} | 11.9 | (2.0) |
| Throat (%) | 14.0 | (1.7) | 13.9 | (1.4) | 14.1 | (2.3) |
| Total NT (%) | 31.4 | (1.2) | 38.3 | (2.4) | 33.3 | (4.3) |
| Tracheal Filter (%) | 45.2 | (2.2) | 47.0 | (0.2) | 46.7 | (1.4) |

Mean values with standard deviations (SD) shown in parenthesis, n = 3.
All experiments used 5 actuations.
^{a}p < 0.05 significant effect of formulation on deposition region (one-way ANOVA).
^{b}p < 0.05 significant difference compared to AS formulation (post-hoc Tukey).

The best configurations for the surfactant powder formulations were then tested for aerosol size distribution using the NGI experimental setup. Table 11 compares the characteristics of the aerosol exiting the air-jet DPI as well as the NGI pre-separator fraction. The AS-EEG formulation had the lowest pre-separator fraction of ~8% and mean FPF_{5 μm} and FPF_{1 μm} values of ~85% and 25%, respectively. Surprisingly, despite similar lung delivery performance with the NT model, the surfactant formulations had significantly different aerosol characteristics with smaller FPFs and larger pre-separator fractions. The Survanta-EEG formulation produced the largest aerosol sizes as indicated by the largest NGI pre-separator fraction of nearly ~42% and lowest FPFs (FPF_{5 μm}≈43% and FPF_{1μm}≈11%). The DPPC-BYL3-EEG formulation produced midrange sizes with a pre-separator fraction of ~30% and a mean FPF_{5 μm} and FPF_{1 μm} of ~60% and ~16%, respectively.

TABLE 11
Aerosol characterization based on next generation impactor
(NGI) testing for each EEG formulation with 10 mg powder
loadings and the best metering clement configuration
(aerosol at air-jet DPI device outlet).

| | | | Survanta- | | DPPC- | |
|---|---|---|---|---|---|---|
| Deposition Region | AS-EEG | | EEG | | BYL3-EEG | |
| DPI Retention (%)^{a} | 16.5 | (1.5) | 12.9 | (2.6) | 9.1 | (0.6)^{b} |
| Pre-separator Fraction (%)^{a} | 8.3 | (1.0) | 41.6 | (2.4)^{b} | 30.2 | (1.2)^{b} |
| FPF_{5 μm} (%)^{a} | 84.7 | (2.1) | 43.0 | (2.6)^{b} | 60.1 | (1.6)^{b} |
| FPF_{1 μm} (%)^{a} | 24.9 | (2.1) | 10.5 | (2.0)^{b} | 15.6 | (0.5)^{b} |

Mean values with standard deviations (SD) shown in parenthesis, n = 3.
All experiments used 5 actuations.
^{a}p < 0.05 significant effect of formulation on deposition region (one-way ANOVA).
^{b}p < 0.05 significant difference compared to AS formulation (post-hoc Tukey).

Two passive metering elements, the shelf volume and the PDT diameter, were identified in this Example and enabled the air-jet DPI to be tuned across different powder formulations. Initial tuning of the shelf volume was investigated based on the powder density. Since the Survanta-EEG formulation had a higher bulk density, a smaller shelf volume was initially used for tuning. Reducing the shelf volume incrementally showed improved performance up to a certain point. The decreased volumes (~20 and ~16 μL) improved performance, after which performance dropped at ~8 μL. It is likely the aerosolization chamber needs a minimum shelf volume between 8-16 μL for effective aerosolization. A secondary set of screenings focused on the PDT diameter while maintaining a shelf volume between 8-16 L. A final configuration with a shelf volume of 12 μL and PDT diameter of 1.5 mm was found to produce the target performance. When the shelf volume was increased for the DPPC-BYL3-EEG formulation (due to lower bulk density), performance did not improve, indicating the shelf geometry will impede effective aerosolization beyond a certain volume (between 26-40 μL), and 10 actuations were needed to empty the device. Considering the PDT, increasing the diameter from 3 to 3.5 mm maintained the high lung delivery efficiency (with more consistency) while significantly decreasing the required number of actuations to empty. With a low density powder (~0.04 g/cm³), a larger opening was needed to aid the powder transfer from reservoir to aerosolization chamber. Careful tuning of the shelf volume and PDT diameter should allow for optimal performance with each formulation, and, in this Example, tuning of these passive metering elements allowed for two surfactant-EEG powder formulations to perform similarly to the previous best-case AS-EEG formulation.

Two insights from this Example including lowering the shelf volume based on formulation density (with an estimated shelf volume limit equivalent to <3 mg of powder being ideal for some embodiments) and modifying the PDT diameter based on the density of the powders (increasing the opening when <0.10 g/cm³ or decreasing when >0.10 g/cm³).

During in vitro testing using a preterm infant NT model, the two additional surfactant based formulations (Survanta-EEG and DPPC-BYL3-EEG) were found to perform similar to the model AS-EEG formulation when the metering elements were tuned, both providing a >45% lung delivery efficiency. Two general metrics for best performance included a shelf volume limit equivalent to <3 mg of powder and a decrease or increase of the PDT diameter for higher or low density formulations, respectively (compared to ~0.10 g/cm³).

### Example 9. Cyclic Loading Device with Powder Side Entry to Aerosolization Chamber

Aerosol sizing experiments were conducted at two dose loading levels (10 and 30 mg) using a cyclic loading device with a side powder delivery tube (PDT; access port). The cyclic loading device corresponded with FIGS. 6C and 6D and their accompanying descriptions above. As illustrated in FIG. 6C, the cyclic loading device was constructed with a reservoir on the side of the aerosolization chamber and an inclined PDT leading to a lower section of the aerosolization chamber. The PDT was angled and sized such that powder flowed into the aerosolization chamber until it reached the top of the PDT opening, and then stopped flowing, thus metering the initial dose for aerosolization. The PDT had a diameter of 3.5 mm and an angle of 35 degrees from horizontal. The inclined cut of the PDT entering the aerosolization chamber resulted in an elliptical shape opening into the aerosolization chamber. The profile shape of the reservoir, PDT, and lower aerosolization chamber section was such that the powder could flow freely, without experiencing constrictions that would limit the free flow of powder due to gravity. For the device tested, the “metered powder % volume”=“metered powder volume”/“total aerosolization chamber volume”=0.213 mL/0.575 mL=37%. The device also included a 67 mm long exit capillary.

To determine aerosol size, the device was loaded with 10 or 30 mg of albuterol sulfate (AS) excipient enhanced growth (EEG) formulation, similar with the case studies described above (Howe et al., 2023). The outlet capillary was connected to a Next Generation Impactor (NGI; including preseparator) using a custom connector that enabled the entry of co-flow air for steady airflow through the NGJ. Each device was actuated five times for consistency with Howe et al., (2023) Table 11, using 10 mL volumes of air delivered at 3 L/min each. A downstream vacuum pump pulled air through the NGI and custom adaptor enabling a constant 45 L/min flow rate through the NGI. Fine particle fraction (FPF) values were calculated based on the methods described by Howe et al. (2023) and based on the device emitted dose. For the 10 mg initial dose loading and five actuations, aerosol mass median aerodynamic diameter (MMAD), FPF<5 μm, and FPF<lpm, were 1.57 μm, 89.9% and 21.1%, respectively. For the 30 mg initial dose loading and five actuations, aerosol mass median aerodynamic diameter (MMAD), FPF<5 μm, and FPF<lpm, were 1.55 μm, 95.0% and 21.6%, respectively. For both 10 mg and 30 mg loading cases, the side access port cyclic loading device produced a high quality aerosol of at least as good of parameters as observed from top access port devices (see preceding Examples). Emitted doses remained above 50% and may be improved with further optimization of the inclined angle (e.g., >35 degrees) and PDT opening size.

### Example 10. Elimination of Reservoir and Metering System

The same device used in Example 9 above was compared with a variant which had PDT and reservoir system removed. The PDT opening was replaced with a smoothly curving wall surface. The aerosolization chamber was filled with as much AS-EEG formulation as possible (35 mg), which was still below the level of the inlet jet centerline. Performance experiments were conducted as described above with the device actuated five times into the NGJ. Emptying of the variant device was very poor (14% emitted dose). Based on the extremely poor device emptying (much less than 50% of the aerosol) sizing of the aerosol is not of consequence.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be separated from or combined with the features of any of the other several embodiments without departing from the scope or spirit of the present invention. While the listed components of illustrated embodiments are exemplary, some embodiments may use fewer components, add additional components, or interchange components. Any recited method can be carried out in the order of events recited or in any other order which is logically possible.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are described.

What is claimed is:

1. A method of delivering a full dose of powder to a patient using a dry powder inhaler (DPI), comprising repeating for consecutive cycles
pre-actuation, metering an amount of powder from a reservoir through an opening into an aerosolization chamber by positioning the reservoir above a powder support surface of the aerosolization chamber with respect to gravity so that a metering space between the powder support surface and the opening fills with powder of an amount less than the full dose;
actuating the DPI so that one or more air jets pass through the aerosolization chamber to aerosolize the powder; and
transporting the aerosolized powder from the aerosolization chamber to airways of the patient,
wherein the amount of powder metered and emitted as an aerosol is consistent across multiple of the consecutive cycles, and
wherein at least one jet of the one or more air jets is positioned to not impinge on the powder in the metering space.

2. The method of claim 1, wherein the actuating step is performed by one or more of (i) active actuation by a positive pressure air source and (ii) passive actuation by user inhalation.

3. The method of claim 1, wherein the opening is arranged at a top of the aerosolization chamber.

4. The method of claim 1, wherein the opening is arranged at a side of the aerosolization chamber.

5. The method of claim 1, wherein the one or more air jets further comprise at least a second jet which enters the metering space to impinge on the powder in the metering space.

6. The method of claim 1, further comprising adjusting a total volume of the reservoir to reduce or eliminate dead space.

7. The method of claim 1, wherein the powder support surface is a top surface of a shelf inside the aerosolization chamber, wherein the shelf is positioned between a ceiling and floor of the aerosolization chamber.

8. The method of claim 1, wherein the aerosolization chamber comprises enclosing walls with one or more inlet orifices and one or more air outlet orifices, wherein the enclosing walls are continuously curving with no sharp corners with the exception of orifice openings of the one or more inlet orifices and the one or more outlet orifices and the opening for powder loading.

9. The method of claim 1, wherein the aerosolization chamber comprises one or more inlet orifices and one or more outlet orifices, and wherein a portion of the aerosolization chamber exists on either side of the one or more inlet orifices and at least one outlet orifice of the one or more outlet orifices.

10. A dry powder inhaler (DPI), comprising
an aerosolization chamber comprising a powder support surface;
a reservoir configured to hold at least a full dose of powder to be delivered to a patient;
an opening configured to permit powder from the reservoir to fall into the aerosolization chamber because of gravity;
a metering space between the powder support surface and the opening configured to fill with powder of an amount less than the full dose in the reservoir when the reservoir is positioned above the powder support surface with respect to gravity; and
one or more air inlet orifices to the aerosolization chamber, wherein each of the one or more inlet orifices admits an air jet into the aerosolization chamber, wherein at least one inlet orifice of the one or more inlet orifices is positioned so that an initial air jet from the at least one inlet does not enter the metering space.

11. The DPI of claim 10, wherein the opening is arranged at a top of the aerosolization chamber.

12. The DPI of claim 10, wherein the opening is arranged at a side of the aerosolization chamber.

13. The DPI of claim 10, wherein the one or more air inlet orifices comprise a first inlet orifice positioned so that an air jet from the first inlet orifice does not enter the metering space and a second inlet orifice positioned so that an air jet from the second inlet orifice does enter the metering space.

14. The DPI of claim 10, further comprising a shelf of which the powder support surface is a top surface, wherein the shelf is positioned between a ceiling and floor of the aerosolization chamber.

15. The DPI of claim 10, wherein the aerosolization chamber comprises enclosing walls which include the one or more inlet orifices and one or more outlet orifices, wherein the enclosing walls are continuously curving with no sharp corners with the exception of the one or more air inlet orifices and the one or more outlet orifices and the opening for powder loading.

16. The DPI of claim 10, wherein the reservoir has an adjustable total volume.

17. The DPI of claim 10, further comprising at least one outlet orifice, wherein a portion of the aerosolization chamber exists on either side of the one or more inlet orifices and the at least one outlet orifice.

18. A platform, comprising
a DPI according to claim 10; and
a patient interface.

19. The platform according to claim 18, further comprising a positive pressure air source configured for actuating the DPI so that one or more air jets pass through the aerosolization chamber to aerosolize the powder.
